(19) United States
(12) Patent Application Publication
(10) Pub. No.: US 2019/0320977 A1
(43) Pub. Date: Oct. 24, 2019

(54) ORAL APPLIANCE MONITORING

(71) Applicant: Oventus Medical Limited, Indooroopilly, Queensland (AU)

(72) Inventors: Neil ANDERSON, Roseville, New South Wales (AU); Christopher Patrick HART, Indooroopilly, Queensland (AU); Anthony IGNACIO, Bossly Park, New South Wales (AU); Zoran MILIJASEVIC, Bayview, New South Wales (AU); Gaetano Dario GARGIULO, Kogarah, New South Wales (AU)

(21) Appl. No.: 16/465,023

(22) PCT Filed: Nov. 29, 2017
(86) PCT No.: PCT/AU2017/051316

§ 371 Date: May 29, 2019

(30) Foreign Application Priority Data
Nov. 30, 2016 (AU) 2016904924
Jun. 1, 2017 (AU) 2017902091

(51) Int. Cl. A61B 5/00; A61B 5/08

(52) U.S. Cl. CPC A61B 5/682; A61B 5/082; A61B 5/0878; A61B 5/6885; A61B 5/6843; A61B 5/684

(57) ABSTRACT

A system monitors use of an oral appliance including a body positioned within an oral cavity of the user in use. The system includes an appliance monitoring device attached to or embedded within the oral appliance. The appliance monitoring device includes at least one sensor including a pressure sensor generating a signal indicative of a pressure, a data store, and a processing device analysing signals from the at least one sensor to determine a usage state. In response to a determination that the appliance is in use, the processor generates usage data at least partially indicative of the use and stores the usage data in the data store. The system also includes one or more processing systems acquiring the usage data from the monitoring device and storing an indication of the usage data and/or causing a representation to be displayed at least partially in accordance with the usage data.

### BACKGROUND OF THE INVENTION

The present invention relates to a system and method for monitoring use of an oral appliance, and in one particular example to a system and method for monitoring compliance and/or respiratory data associated with use of a breathing assistance appliance.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Poor quality or ineffective breathing is an issue which can affect the performance of people in their day to day activities either while they are awake and/or when they are asleep. While awake this can be less optimal performance in activities such as sport or even while performing everyday tasks. While asleep breathing disorders can lead to snoring and/or sleep apnoea.

Current therapy for treatment of OSA can include lifestyle changes, the use of mechanical devices, such as oral or nasal devices that augment the airway, surgical procedures to enlarge and stabilize the airway during sleep, and continuous or variable positive airway pressure (CPAP, VPAP) devices.

However, surgical procedures can be severe and are not therefore widely used unless absolutely necessary. Whilst CPAP and VPAP devices have had a positive impact, these can be uncomfortable to wear for prolonged time periods, are expensive, and are often noisy, which can in turn lead to additional sleep disturbance. As a result, surgery, VPAP and CPAP treatment have limited application in treating sleep apnoea, and are not generally considered appropriate treatment for snoring.

CPAP masks suffer from several drawbacks including leakage and discomfort and often users experience a degree of claustrophobia whilst wearing the mask. Furthermore, as CPAP systems must supply air at sufficient pressure to maintain an airway and act as a pneumatic splint, relatively high pressures are typically required. In addition, high flow rates are required as the mask supplies all of the air for a user during inhalation. In order to achieve such high pressures and flow, relatively large and noisy pumps such as air blowers are conventionally used.

However, even in non CPAP related appliances, users often fail to comply with recommend usage, which in turn can lead to reduced improvement in health outcomes. Additionally, even when used, it is difficult to quantify the benefit provided, which can in turn lead to a further reduced motivation to comply with usage recommendations.

One attempt to address this issue is described in CA2829973, which discloses a method and an apparatus for verifying compliance with a dental appliance therapy for a human patient is described. In this document a temperature and spatial orientation of an appliance are periodically measured and compliance with the dental appliance therapy is determined by performing a spectral analysis of the measured parameter. However, this arrangement is complex and does not always accurately detect compliance.

WO2012155214 and WO2015149127 describe a breathing assistance apparatus including a body for positioning within an oral cavity of a user, the body defining at least one first opening for allowing airflow between lips of the user, two second openings provided in the oral cavity to allow air flow into and out of a posterior region of the oral cavity and two channels, each channel connecting a respective second opening to the at least one first opening.

### SUMMARY OF THE PRESENT INVENTION

In one broad form an aspect of the present invention seeks to provide a system for monitoring use of an oral appliance, the oral appliance including a body that is positioned within an oral cavity of the user in use, the system including: an appliance monitoring device which in use is attached to or embedded within the oral appliance, the appliance monitoring device including: at least one sensor, the at least one sensor including a pressure sensor that generates a signal indicative of a pressure; a data store; and, a processing device that: analyses signals from the at least one sensor to determine a usage state; in response to a determination that the appliance is in use, generates usage data at least partially indicative of the use; and, stores the usage data in the data store; one or more processing systems that: acquire the usage data from the monitoring device; and, at least one of: store an indication of the usage data; and, cause a representation to be displayed at least partially in accordance with the usage data.

In one embodiment the appliance monitoring device includes at least one of: a clock, the clock generating an indication of at least one of a time and a date; a physical connection that connects to the one or more processing systems, the processing device operating to transfer the usage data to the one or more processing systems via the connection; a transmitter, the processing device operating to transmit the usage data to the one or more processing systems using the transmitter; and, a power supply that powers the at least one sensor and the processing device.

In one embodiment the appliance monitoring device includes a housing containing the at least one sensor and the processing device, the housing being removably mounted to the body.

In one embodiment the appliance monitoring device includes: a sensor housing containing the at least one sensor; a processing device housing containing the processing device; and, at least one electrical connection extending between the sensor and processing device housings.

In one embodiment the appliance monitoring device includes a housing mounted to the body such that the housing is outside of the oral cavity in use.

In one embodiment the pressure sensor measures at least one of: an air pressure in an airway of the oral appliance; an air pressure in an airway of the user; a contact between the user and the appliance; and, a contact pressure of contact between the user and the appliance.

In one embodiment the at least one sensor includes: a first pressure sensor that measures at least one of a contact or contact pressure; a second pressure sensor that measures an air pressure in an airway; an oxygen sensor that senses oxygen levels in exhaled air; a carbon dioxide sensor that senses carbon dioxide levels in exhaled air; a temperature sensor that measures a temperature; a moisture sensor that measures a moisture; a humidity sensor that measures a humidity; and, a movement sensor that measures at least one of: a position of the oral appliance; an orientation of the oral appliance; and, a movement of the oral appliance.

In one embodiment the usage data includes at least one of: an identifier indicative of at least one of an identity and type of the oral appliance; a time the signals were measured; a date on which the signals were measured; an indication of a usage state; sensor data indicative of signals from the sensors; at least one parameter at least partially derived using signals from the sensors; compliance data indicative of a compliance period associated with a period of use of the oral appliance by a user; respiratory device data indicative of at least one operational characteristic of a respiratory device; respiratory data indicative of at least one respiratory characteristic of the user of the oral appliance; and, sleep data indicative of at least one sleep characteristic of the user of the oral appliance.

In one embodiment the representation is indicative of at least one of: signals from the at least one sensor; changes in at least one parameter over time; comparison of the signals to one or more thresholds; and, comparison of the at least one parameter to one or more thresholds.

In one embodiment the monitoring device at least partially processes the sensor signals by at least one: filtering the signals; amplifying the signals; digitizing the signals; and, parameterizing the signals.

In one embodiment the processing device determines if the appliance is in use based on a signal from a pressure sensor indicative of a contact between the user and the appliance.

In one embodiment: if an appliance is in use, the processing device at least one of: selectively updates compliance data indicative of a compliance period; and, records sensor data indicative of signals from the sensors. If an appliance is not in use, the processing device: uses signals from the at least one sensor to generate a reference; and, stores an indication of the reference in the data store.

In one embodiment if the appliance is in use, the processing device: uses usage data to determine if a compliance period is underway; and, if a compliance period is underway: determines if at least one of a pressure determined by a pressure sensor and a temperature determined by a temperature sensor exceed a respective reference; and, in response to a successful determination, updates compliance data to extend the compliance period; if a compliance period is not underway: monitors for a change in at least one of a pressure determined by a pressure sensor and a temperature determined a temperature sensor; and, in response to a change, updates compliance data to commence a compliance period.

In one embodiment the change corresponds to at least one of: spikes; changes having a magnitude greater than a threshold; and, changes having a rate of change greater than a threshold.

In one embodiment the processing device uses a usage state to at least partially control operation of the system.

In one embodiment the processing device uses the usage state to control a signal sampling rate.

In one embodiment the processing device: exits a low power mode; determines the usage state; optionally generates usage data; and, returns to the low power mode for a defined time interval.

In one embodiment the defined time limit is set in accordance with the usage state.

In one embodiment the system: analyses sensor data from the at least one sensor to determine at least one of: a temperature in an airway; an air pressure in an airway; uses at least one of the temperature and air pressure to monitor at least one of: respiratory device data indicative of at least one operational characteristic of a respiratory device; respiratory data indicative of at least one respiratory characteristic of the user of the oral appliance; and, sleep data indicative of at least one sleep characteristic of the user of the oral appliance.

In one embodiment the system analyses sensor data from the at least one sensor to determine at least one of: a respiration rate; a respiration magnitude; and, a degree of snoring.

In one embodiment the system is used to perform a sleep test and wherein the pressure sensor includes an air pressure sensor that measures an air pressure in an airway of the oral appliance or a connector system, and wherein the sensor data is used to generate at least one of: respiratory data indicative of at least one respiratory characteristic of the user of the oral appliance; and, sleep data indicative of at least one sleep characteristic of the user of the oral appliance.

In one embodiment the one or more processing systems: acquire user sensor data indicative of signals from at least one user sensor; and, generate sleep data at least partially indicative of a sleep characteristic of a user of the oral appliance at least in part using the usage data and the user sensor data.

In one embodiment the at least one user sensor includes at least one of: an oxygen sensor that senses oxygen levels in exhaled air; a carbon dioxide sensor that senses carbon dioxide levels in exhaled air; respiratory sensors that sense a respiratory effort or rate; pulse oximetry sensor that measures a blood oxygen level; an ECG sensor; an EEG sensor; and, a heart rate sensor that measures a heart rate.

In one embodiment the oral appliance includes at least one bite member coupled to the body, the bite member being positioned at least partially between the user's teeth and the body in use, and wherein the at least one sensor includes a pressure sensor that senses contact of the user's teeth with the bite member based on a pressure between the body and bite member.

In one embodiment oral appliance includes first and second bodies, the first body includes an adjustable mounting configured to interconnect the first and second bodies to thereby allow a relative position of the first and second bodies to be adjusted, and wherein the at least one sensor includes a pressure sensor configured to determine a relative pressure between the first and second bodies.

In one embodiment the oral appliance includes an extra-oral opening for allowing airflow between lips of the user and wherein the at least one sensor monitors at least one of an air pressure and a temperature in the extra-oral opening.

In one embodiment the oral appliance includes at least one extra-oral opening defined by a tubular body protruding from the appliance and wherein the at least one sensor includes a pressure sensor that senses a contact between a user's lips and an external surface of the tubular body.

In one embodiment the oral appliance includes at least one extra-oral opening in fluid communication with at least one intra-oral opening via a channel, the intra-oral opening being provided in the oral cavity to direct airflow into and/or out of a posterior region of the oral cavity, and wherein the at least one sensor monitors at least one of an air pressure and a temperature in the channel.

In one embodiment the body defines at least two channels, each channel connecting an intra-oral opening to at least one extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

In one embodiment the oral appliance includes at least one extra-oral connector and a connector system connected to the at least one extra-oral connector, the connector system including at least one passageway for allowing airflow through at least one of the oral appliance and nose of the user, and wherein the at least one sensor monitors at least one of an air pressure and a temperature in the at least one passageway.

In one embodiment the passageway is connected to a positive airway pressure (PAP) device, and wherein the at least one sensor monitors operation of the PAP device.

In one embodiment air from the PAP device is delivered to the user through at least one of: the nose of the user; and, the oral appliance.

In one embodiment the oral appliance includes at least one of: a valve; a restrictor; and, a heat and moisture exchanger.

In one embodiment the oral appliance includes a number of ports to allow for inhalation or exhalation, wherein at least one port includes a valve to control a flow restriction.

In one embodiment the system includes at least two air pressure sensors for measuring nasal and oral breathing respectively.

In one broad form an aspect of the present invention seeks to provide a breathing assistance system including: an oral appliance, the oral appliance including a body that is positioned within an oral cavity of the user in use; an appliance monitoring device which in use is attached to or embedded within the oral appliance, the appliance monitoring device including: at least one sensor, the at least one sensor including a pressure sensor that generates a signal indicative of a pressure; a data store; and, a processing device that: analyses signals from the at least one sensor to determine a usage state; in response to a determination that the appliance is in use, generates usage data at least partially indicative of the use; and, stores the usage data in the data store; one or more processing systems that: acquire the usage data from the monitoring device; and, at least one of: store an indication of the usage data; and, cause a representation to be displayed at least partially in accordance with the usage data.

In one broad form an aspect of the present invention seeks to provide an appliance monitoring device which in use is attached to or embedded within an oral appliance, the appliance monitoring device including: at least one sensor, the at least one sensor including a pressure sensor that generates a signal indicative of a pressure; a data store; and, a processing device that: analyses signals from the at least one sensor to determine a usage state; in response to a determination that the appliance is in use, generates usage data at least partially indicative of the use; and, stores the usage data in the data store.

In one broad form an aspect of the present invention seeks to provide a method for monitoring use of an oral appliance, the oral appliance including a body that is positioned within an oral cavity of the user in use, the method including: providing an appliance monitoring device which is attached to or embedded within the oral appliance, the appliance monitoring device including: at least one sensor, the at least one sensor including a pressure sensor that generates a signal indicative of a pressure; and, a data store; and, a processing device; using the processing device to: analyse signals from the at least one sensor to determine a usage state; in response to a determination that the appliance is in use, generate usage data at least partially indicative of the use; and, store the usage data in the data store; using one or more processing systems to: acquire the usage data from the monitoring device; and, at least one of: store an indication of the usage data; and, cause a representation to be displayed at least partially in accordance with the usage data.

In one broad form an aspect of the present invention seeks to provide a system for monitoring a user of an oral appliance, the oral appliance including a body that is positioned within an oral cavity of the user in use, the system including: an appliance monitoring device which in use is attached to or embedded within the oral appliance, the appliance monitoring device including: at least one sensor, the at least one sensor including an air pressure sensor that generates a signal indicative of an air pressure; a data store; and, a processing device that: receives signals from the at least one sensor; and, stores sensor data indicative of signals from the sensors in the data store; one or more processing systems that: acquire the sensor data from the appliance monitoring device; and, use the sensor data to generate at least one of: respiratory data indicative of at least one respiratory characteristic of the user of the oral appliance; and, sleep data indicative of at least one sleep characteristic of the user of the oral appliance.

In one embodiment the system includes a user monitoring device including at least user one sensor for monitoring an attribute of a user, and wherein the one or more processing systems: acquire user sensor data indicative of signals from at least one user sensor; and, generate sleep data at least partially indicative of a sleep characteristic of a user of the oral appliance at least in part using the sensor data and the user sensor data.

In one embodiment the at least one user sensor includes at least one of: an oxygen sensor that senses oxygen levels in exhaled air; a carbon dioxide sensor that senses carbon dioxide levels in exhaled air; respiratory sensors that sense a respiratory effort or rate; pulse oximetry sensor that measures a blood oxygen level; an ECG sensor; an EEG sensor; and, a heart rate sensor that measures a heart rate.

In one embodiment the system is used to perform a sleep test.

In one broad form an aspect of the present invention seeks to provide a method for monitoring a user of an oral appliance, the oral appliance including a body that is positioned within an oral cavity of the user in use, the method including: providing an appliance monitoring device which in use is attached to or embedded within the oral appliance, the appliance monitoring device including: at least one sensor, the at least one sensor including an air pressure sensor that generates a signal indicative of an air pressure; a data store; and, a processing device; using the processing device to: receive signals from the at least one sensor; and, store sensor data indicative of signals from the sensors in the data store; using one or more processing systems to: acquire the sensor data from the appliance monitoring device; and, use the sensor data to generate at least one of: respiratory data indicative of at least one respiratory characteristic of the user of the oral appliance; and, sleep data indicative of at least one sleep characteristic of the user of the oral appliance.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:—

FIG. 1 is a schematic diagram of an example of a system for monitoring use of an oral appliance;

FIG. 2 is a flow chart of an example of a method for monitoring use of an oral appliance;

FIG. 3 is a schematic diagram of a further example of a system for monitoring use of an oral appliance;

FIG. 4 is a schematic diagram of an example of a processing system of FIG. 3;

FIG. 5 is a schematic diagram of an example of a client device of FIG. 3;

FIG. 6A is a schematic perspective top view of an example of an oral appliance;

FIG. 6B is a schematic perspective underside view of the oral appliance of FIG. 6A;

FIG. 6C is a schematic front view of the oral appliance of FIG. 6A;

FIG. 6D is a schematic rear view of the oral appliance of FIG. 6A;

FIG. 6E is a schematic cut away view of a first example of an appliance monitoring device;

FIG. 6F is a schematic cut away view of a first example of an appliance monitoring device;

FIG. 6G is a schematic cut away view of an appliance including two monitoring devices;

FIG. 7A is a schematic top side perspective view of an example of an oral appliance including an appliance monitoring device;

FIG. 7B is a schematic under side perspective view of the oral appliance of FIG. 7A;

FIG. 7C is a schematic top side perspective view of the oral appliance of FIG. 7A with the appliance monitoring device removed;

FIG. 7D is a schematic cut away front view of the oral appliance of FIG. 7A with the appliance monitoring device removed;

FIG. 7E is a schematic perspective top side view of the oral appliance monitoring device;

FIG. 7F is a schematic cut away side view of the oral appliance of FIG. 7A;

FIG. 8 is a flow chart of an example of a process for registering an appliance with a user;

FIG. 9 is a flow chart of a first specific example of a process for monitoring use of an oral appliance;

FIG. 10 is a flow chart of a second specific example of a process for monitoring use of an oral appliance;

FIG. 11 is a graph showing an example of normalised temperature and pressure changes when monitoring the use of an oral appliance;

FIG. 12 is a flow chart of an example of a process for analysing compliance relating to the use of an oral appliance;

FIG. 13 is a flow chart of an example of a process for analysing sensor data relating to the use of an oral appliance;

FIG. 14A is a schematic perspective top side view of a further example of a system for providing breathing assistance;

FIG. 14B is a schematic perspective rear side view of the connector system used in the system of FIG. 14A;

FIG. 14C is a schematic perspective top side view of the oral appliance used in the system of FIG. 14A;

FIG. 14D is a schematic perspective view of a second example of an oral appliance used in the system of FIG. 14A showing a connector system connected to an extra-oral opening of the appliance;

FIG. 15A is a schematic perspective view of a further example of a system for providing breathing assistance having a further example of a connector system;

FIG. 15B is a schematic front perspective view of a nasal connecting portion with nasal pillows for use with the connector system of FIG. 15A;

FIG. 16A is a schematic perspective view of a further example of a connector system;

FIG. 16B is a schematic side cut-away view of the connector system of FIG. 16A.

FIG. 17A is a schematic perspective view of an example of a connector system;

FIG. 17B is a schematic side cut-away view of the connector system of FIG. 17A;

FIG. 17C is a schematic rear view of the connector system of FIG. 17A;

FIG. 18 is an example of air pressure variations measured for oral and nasal passageways during breathing;

FIG. 19A is a schematic perspective view of a further example of a system for providing breathing assistance;

FIG. 19B is a schematic side view of the system of FIG. 19A;

FIG. 20A is a schematic perspective view of a further example of a system for providing breathing assistance;

FIG. 20B is a schematic side view of the system of FIG. 20A; and,

FIG. 20C is a schematic plan view of the system of FIG. 20A.

### DETAILED DESCRIPTION OF THE PREFERRED EMBODIMENTS

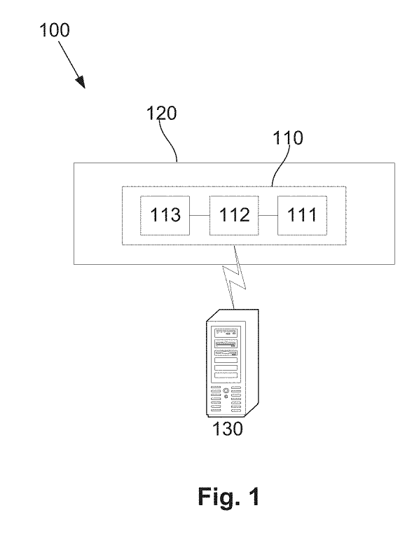

An example of a system for monitoring the use of an oral appliance will now be described with reference to FIG. 1.

In this example, the system includes an appliance monitoring device 110 which in use is attached to or embedded within the oral appliance 120. The oral appliance could be of any appropriate form but typically includes a body that is positioned within an oral cavity of the user in use. The oral appliance may define one or more airways allowing airflow into or out of the user's mouth to facilitate breathing and/or to allow application of positive airway pressure (PAP). Example oral appliances will be described in more detail below but it will be appreciated from the following description that the system can be used with a wide range of different oral appliances and the specific examples described herein are not intended to be limiting.

The appliance monitoring device 110 includes an electronic processing device 111, a data store 112 and one or more sensors 113. The one or more sensors 113 include at least a pressure sensor that generates a signal indicative of a pressure. The pressure signal could be indicative of a contact pressure within or on a surface of the oral appliance, or could be indicative of an air pressure as will be described in more detail below.

In use, the electronic processing device is adapted to acquire signals from the sensors and then optionally process these and store usage data based on the signals in the data store 112, allowing this to be subsequently retrieved and used as required. It will be appreciated from this that the processing device can be a standard microprocessor, although this is not essential and any suitable arrangement, such as a microchip processor, logic gate configuration, firmware optionally associated with implementing logic such as an FPGA (Field Programmable Gate Array), or any other electronic device, system or arrangement, could be used.

The appliance monitoring device 110 is also typically in communication with one or more processing systems 130 which can be at least partially used in monitoring use of the oral appliance. The processing systems 130 could be of any suitable form and could include computer systems such as personal computers, laptops, desktops or servers, or mobile communication devices such as smart phones or tablets or the like. The appliance monitoring device 110 and processing systems 130 can be configured to communicate via a wired or wireless connection, including via intervening network architectures, depending upon preferred implementation and examples of this will be described in more detail below.

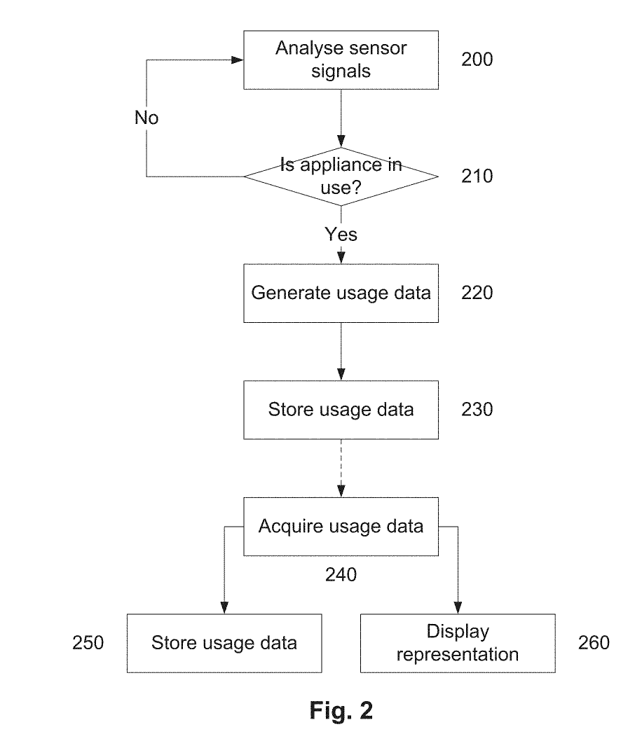

An example of the process of monitoring the use of an oral appliance will now be described with reference to FIG. 2.

In this example, at step 200 the processing device 111 analyses signals from the one or more sensors 113. The processing device uses signals from the sensors to determine a usage state which is in turn indicative of whether or not the oral appliance is in use. It will be appreciated that this could be achieved in a number of ways depending on the preferred implementation and the nature of the sensors 113. For example, if the pressure sensor is configured to detect a contact pressure, the presence of a contact pressure alone could be sufficient to indicate the oral appliance is being used. Alternatively, the pressure sensor could be used to detect an air pressure, with an elevated air pressure within the appliance being indicative of breathing and hence use.

If it is determined that the oral appliance is not in use at step 210, monitoring can continue by returning to step 200. Otherwise, at step 220, in response to determination that the appliance is in use, the processing device generates usage data at least partially indicative of the use, with the usage data being stored in the data store 112 at step 230.

The usage data can take on any one of a number of forms and could include sensor data indicative of signals received from the sensors, parameters derived from the sensor signals, such as frequency components, or signal magnitudes, or could include an indication that the device is in use, and optionally a duration of use.

In one example, this process is performed to allow the appliance monitoring device to remain in a low power mode for a majority of the time, periodically waking and monitoring signals to determine if the device is being used. This can be used to reduce power consumption, although this is not essential and alternatively monitoring could be performed continuously. Additionally, this arrangement allows usage data to be recorded only when the device is being used, thereby reduce storage requirements.

Subsequent to this, the usage data is acquired by the processing system 130, for example by having the processing system communicate with the processing device 111 of the appliance monitoring device 110 and retrieve the data therefrom. Alternatively usage data could be pushed from the appliance monitoring device 110 to the processing system 130.

Having acquired usage data at step 240, the processing system 130 can then operate to either store usage data in a memory or other data store, such as a database, allowing this to be subsequently retrieved and used as required. Additionally and/or alternatively, the processing system 130 can organise to display a representation generated at least partially in accordance with the usage data at step 260. The representation could be of any appropriate form and could include an indication of compliance with an intended use of the oral appliance, information regarding respiration of the user or operation of a respiratory device connected to the oral appliance, or the like.

Accordingly, it will be appreciated that the above described system utilises an appliance monitoring device including one or more sensors, which are coupled to or embedded within the oral appliance, to thereby monitor use of the oral appliance. In particular, this involves using a pressure sensor, which can detect changes in pressure resulting either from contact of the user with the oral appliance, or changes in air pressure, for example within an airway of either a user or the oral appliance, thereby allowing use of the oral appliance to be monitored. Using pressure sensors to monitor usage provides a cheap and straightforward mechanism to detect usage, as well as allowing this to be used in detecting respiratory data relating to breathing of the user.

Usage data indicative of the use is then stored locally in the appliance monitoring device, allowing this to be subsequently transferred to one or more processing systems for further storage, analysis and/or review. This can in turn be utilised to ensure adherence with compliance requirements associated with the oral appliance and/or to monitor respiration of the user or operation of a respiratory device.

A number of further features will now be described.

In one example, the appliance monitoring device includes one or more of a clock, a physical connection, a transmitter and a power supply. The clock can be used to generate an indication of a time or date, which can in turn be associated with particular usage data, allowing this to be used in ongoing monitoring processes, for example to track compliance with usage requirements.

A physical connection, such as a USB connection, can be provided that connects the one or more processing systems to the processing device, allowing the processing device to transfer usage data to the one or more processing systems via the connection, and also optionally charge the power supply. Alternatively data transfer could be achieved using a wireless transmitter, such as a Bluetooth transmitter, which allows the processing device to transmit the usage data to the one or more processing systems.

The nature of the power supply will vary depending upon the preferred implementation, but typically includes a battery, such as a lithium ion button cell battery or the like. Additionally, the battery could be rechargeable, allowing this to be charged using inductive coupling, physical connection to a charger, or the like.

The appliance monitoring device can include a housing containing the at least one sensor and the processing device, with the housing being removably mounted to the body. Alternatively, the appliance monitoring device could include a sensor housing containing the at least one sensor, a processing device housing containing the processing device and at least one electrical connection, such as wiring, a flexible PCB (printed circuit board) or the like, extending between the sensor and processing device housings. Thus, the appliance monitoring device could include a single housing incorporating all components or multiple housings containing different interconnected components.

Alternatively, the sensor processing device could be directly embedded within oral appliance body, depending on the preferred implementation. In one particular example the housing can be mounted to, or form part of the oral appliance body, such that the housing is outside of the oral cavity in use, although this is not essential and will depend upon the particular configuration and the particular parameters being measured by the sensor. Thus, it will be appreciated that the monitoring device, or components of the monitoring device, such as the one or more sensors, processing device, data store or the like, could be provided in the oral cavity in use. In one example, the housing is made of or contains an epoxy resin material, with components including the sensors, processing device and memory being embedded with the epoxy to protect the components from exposure to moisture or the like.

The pressure sensor could be configured in a number of different ways allowing a variety of different pressures to be measured. This can include measuring an air pressure in an airway of the oral appliance or the user or a contact pressure between the user and the appliance. It will be appreciated that respective pressure sensors may be provided for monitoring different pressures so that for example, a first pressure sensor could be provided which measures a contact or contact pressure with a separate additional second pressure sensor being provided that measures an air pressure in an airway. It will also be appreciated that multiple pressure sensors could of the same type could be provided at different locations within the oral appliance, for example to measure air pressures in different airways, to measure contact pressure with different parts of the user's anatomy, or the like. For example, the monitoring device could be provided on a surface of the appliance at least partially external to the oral cavity in use, thereby allowing contact with a user's lips to be detected, as will be described in more detail below. However, alternatively the monitoring device and/or sensors could be located inside the oral cavity in use, thereby allowing contact with the teeth, tongue or other parts of the oral cavity to be detected.

The monitoring device may also include additional sensors, such as a temperature sensor that measures a temperature, an oxygen sensor that senses oxygen levels in exhaled air; a carbon dioxide sensor that senses carbon dioxide levels in exhaled air; a moisture sensor that measures moisture; a humidity sensor that measures humidity, or a position/movement sensor that measures either a position, orientation or movement of the oral appliance. It will be appreciated from this, that multiple sensors could be provided in multiple different housings, connected to a single processing device and/or other required components, such as a battery, data store and a data interface for allow data to be provided to the one or more processing systems.

The nature of the usage data will vary depending upon the preferred implementation. In one example, the usage data can include any one or more of an indication of a usage state, such as an indication of whether the oral appliance is in use at a particular time, sensor data indicative of signals from the sensors, or one or more parameters at least partially derived from signals from the sensors. Such parameters could include any one or more of compliance data indicative of a compliance period associated with a period of use of the oral appliance by the user, respiratory device data indicative of at least one operation characteristic of a respiratory device, respiratory data indicative of at least one respiratory characteristic of the user of the oral appliance or a sleep parameter indicative of at least one sleep characteristic of the user of the oral appliance.

Thus, in one example, the system may be utilised to monitor compliance and in particular check whether a user is using the oral appliance as intended. This can include ensuring the user is using the oral appliance for a set time, or at particular times, such as when the user is asleep. Additionally or alternatively, the system can be used in order to ascertain other information, such as information regarding the user's respiration, or successful functioning of respiratory devices such as PAP devices attached to the oral appliance.

The usage data may also include other information, such as an identifier indicative of an identity and/or type of a sensor and/or oral appliance. This can be used in order to ensure that collected data is correctly analysed with respect to the particular user of the oral appliance and/or to allow sensors at different locations within the oral appliance to be distinguished. This can be utilised in order to control how the data is analysed, for example analysing data differently depending on the type of oral appliance being used or the location of the sensor within the appliance. The usage data can also include a time or date on which the signals were measured which can be utilised for compliance and logging purposes.

As previously mentioned, the system can be adapted to generate a representation. The representation allows users, or other individuals such as medical practitioners, to review information regarding use of the oral appliance. The representation could simply be an indication of compliance, such as to indicate compliance has been met or not met, but more typically could include information regarding a degree of compliance, such as a number of hours for which the oral appliance has been used during a defined time period. The representation could also be indicative of signals from the at least one sensor, changes in one or more parameters over time, such as changes in a breathing rate or magnitude, comparison of the signals or parameters to one or more thresholds, or the like. This can be used to assist the user and/or medical practitioner understand how the oral appliance is being used and the impact of this upon the user, for example allowing the practitioner to assess whether use of the appliance is assisting the user's breathing and/or sleep, reducing snoring, or the like.

In one example, the processing device can be adapted to at least partially process sensor signals on board the monitoring device. Such processing can be performed by the processing device itself or other suitable electronic components, and can include filtering signals, for example to remove high or low frequency components, amplifying the signals, digitising the signals, parameterising the signals or the like. For example, processing could include performing frequency transformations, such as Fourier transforms, in order to determine frequency components of the signals, which can be utilised in order to identify particular respiratory characteristics, such as a breathing rate, whether the user is snoring or the like. The processing can be performed in order to assist in analysing the signals to determine a usage state, as well as to reduce an amount of data that needs to be stored. Alternatively however raw data could be stored, with processing being performed by the processing system only, once the raw data has been downloaded.

The manner in which a usage state is determined can vary depending on the preferred implementation. In one example, the processing device determines if the appliance is in use at least in part based on a signal from a pressure sensor indicative of a contact between the user and the appliance. However, it will be appreciated that depending on the location and nature of the contact sensor, this doesn't necessarily provide conclusive proof that the oral appliance is in use. For example, similar readings might be obtained if the device is being otherwise handled, or has been inadvertently placed in contact with an object. Accordingly, in one example, contact is used as a first pass indication of whether the appliance is being used, with additional sensing being performed to confirm this is the case.

In one example, this is achieved by monitoring signals from one or more other sensors, such as an air pressure sensor and/or temperature sensor. In this instance, if a pressure or temperature exceeds an ambient pressure or temperature, or the temperatures and pressures are fluctuating, this can be indicative of respiration, and in particular airflow through an airway, and hence is indicative that the device is currently being used. It will be appreciated that in a similar manner, the system can use an increase in temperature and pressure to determine when appliance use commences.

Accordingly, in the above described process, contact is used as a first coarse assessment of use, and in particular to identify if the device is not used, with air pressure and temperature sensing being used to confirm that use is occurring. In this way, compliance can be more accurately tracked.

In one particular example, the processing device is adapted to periodically enter a low powered “sleep” mode, with the processing device waking after a predetermined time period or upon detection of a contact with the user, to thereby reduce power usage. In this instance, when the processing device wakes, the processing device determines if the appliance is in use and if so uses previously stored usage data, and in particular previously stored compliance data, to determine if a compliance period is already underway. This will occur if a compliance period has also already been initiated, for example if the processing device has already determined that the appliance is in use on a previous wake cycle. If a compliance period is underway, the processing device determines if an air pressure or temperature determined by a respective sensor exceeds a respective reference and if so compliance data is updated to extend the compliance period. Thus, if the pressure or temperature exceeds the ambient pressure or temperature measured when the appliance is not in use, this indicates that it is likely that the appliance is currently being used and so that compliance period can be extended. Alternatively, if use is detected and a compliance period has not commenced, the processing device can monitor for a change in pressure or temperature with the change being used to trigger commencement of a compliance period.

In this example, if it is detected that an appliance is in use, the processing device can selectively update compliance data indicative of a compliance period, and/or record sensor data indicative of signals from the sensors. In this regard, if it is determined that the oral appliance is in use this can be used to update compliance data indicative of a total period of time for which the appliance is being used. This can also allow sensor data to be recorded which can be used to determine respiratory, sleep or other relevant parameters. As previously mentioned, recording the sensor data only when the device is in use can reduce power requirements and avoid unnecessary data being recorded.

However, if it is determined that the appliance is not in use, the processing device can still be adapted to use signals from the at least one sensor to generate a reference which is then stored in the data store. In this regard, the reference could correspond to an ambient air pressure and/or temperature which can be further used in order to assess whether a device is in use, as mentioned above.

In one example, the processing device uses the usage state to at least partially control operation of the system. In this regard, the processing device can use the usage state to control a signal sampling rate, for example allowing a lower sampling rate used when the device is not in use, in order to reduce processing and hence power usage requirements. Similarly, the processing device can operate to extend the length of time spent in a sleep mode when the oral appliance is not in use. Thus, in this example, the processing device exists low power mode, determines the usage state optionally generating usage data, for example by updating a compliance data, before returning to the low power mode. The processing device then typically remains in the lower power mode for a defined time interval, with this being set in accordance with the usage state, so that if a device is in use when the low power mode is entered the time period may be shorter than if the device is not in use. This can assist in extending battery life, whilst ensuring that sensor data is collected sufficiently frequently when the appliance is in use.

As previously mentioned, in addition to monitoring a duration of use by recording a compliance period, the system can analyse sensor data from at least one sensor to determine a temperature and/or an air pressure in an airway. The system can then use the temperature or air pressure to generate respiratory device data indicative of at least one operational characteristic of a respiratory device, respiratory data indicative of at least one respiratory characteristic of the user of the oral appliance or sleep data indicative of at least one sleep characteristic of the user of the oral appliance. Thus, in addition to simply monitoring compliance, additional information can be determined such as respiratory or respiratory device data. In one example, such respiratory data can include information regarding one or more of a respiration rate, respiration magnitude, a degree of snoring, or the like.

Additionally, in a further example the one or more processing systems can be adapted to acquire user sensor data indicative of signals from at least one user sensor. The user sensor could be provided as part of a separate sensing device that operates to sense other information regarding the user of the oral appliance. Such user sensors can include any one or more of an oxygen sensor that senses oxygen levels in exhaled air, a carbon dioxide sensor that senses carbon dioxide levels in exhaled air, respiratory sensors that sense a respiratory effort or rate, a pulse oximetry sensor that measures a blood oxygen level, an ECG sensor that measures electrocardiography signals, an EEC sensor that measures electroencephalography signals, or a heartrate sensor that measures a heartrate.

The sensors can be provided as part of or remote to the breathing assistance apparatus. For example, the respiratory sensor could measure tension in an elastic belt extending round the chest or abdomen of the subject, or changes in an inductance of a sensor in a chest strap. Example commercial inductance sensors include Philips Respironics zRIP inductive respiratory effort sensors.

The processing devices can then use the usage data, and in particular the sensor data, and the user sensor data to generate a sleep parameter at least partially indicative of the sleep characteristic of a user of the oral appliance. This can in turn allow a level three sleep assessment to be performed, without requiring the form of monitoring that is typically required. In this particular example, it will be appreciated that the pressure sensor is typically an air pressure sensor that measures an air pressure in an airway of the oral appliance or a connector system coupled thereto, with the sensor data being used to generate respiratory data indicative of at least one respiratory characteristic of the user of the oral appliance and/or sleep data indicative of at least one sleep characteristic of the user of the oral appliance.

As previously mentioned, the appliance sensing device can be used with a wide range of oral appliances. In one example, the oral appliance includes a bite member coupled to the body. The bite member is positioned at least partially between the user's teeth and the body in use and may be customised to fit the particular user. In this example, a contact pressure sensor can be provided that senses contact of the user's teeth with the bite member based on a pressure between the body and the bite member. To achieve this, the sensor can be positioned between the body and the bite member, or within the body and/or bite member, depending on the preferred implementation.

In one example, the oral appliance includes first and second bodies with the first body including an adjustable mounting configured to interconnect the first and second bodies, thereby allowing a relative position of the first and second bodies to be adjusted. In this arrangement, a contact pressure sensor can be configured to determine a relative contact pressure between the first and second bodies, which can in turn be used to detect lateral or longitudinal forces on the user's jaw. This information can be used not only to confirm that the device is being used, but additionally to ensure there is no undue pressure applied to the user's jaws, to ensure a required degree of mandibular advancement is being obtained, as well as being used to detect bruxism.

In one example, the oral appliance includes an extra-oral opening for allowing air flow between lips of the user. The extra-oral opening can open directly into the user's oral cavity or can be connected to a channel extending to intra-oral openings provided towards a rear of the user's oral cavity. This later arrangement is particularly beneficial as it directs air flow into and out of a rear of the user's oral cavity which helps avoid drying of the user's oral cavity. In either case, the sensor(s) can monitor an air pressure and/or temperature in the extra-oral opening, whilst in the latter case the sensor(s) can additionally or alternatively monitor at least one of an air pressure and temperature in the channel.

In one example, the extra-oral opening is defined by a tubular body protruding from the appliance, and in particular between the user's lips. In this example, a contact pressure sensor can be provided that senses a contact between the user's lips and an external surface of the tubular body. In this example, the pressure sensor is provided on an external outer surface of the tubular body and may be embedded in epoxy or the like in order to form part of the external outer surface of the tubular body. In this arrangement a second air pressure sensor can be provided on an internal surface of the body to thereby detect airflow through the opening as previously described.

The oral appliance can also include at least one extra-oral connector and a connector system connected to the at least one extra-oral connector. The connector system can include at least one passageway for allowing air flow through the oral appliance and/or the nose of the user with the sensor(s) monitoring at least one of an air pressure and temperature in the passageway. This can be used in order to allow air flow in to and out of either the oral cavity or the nasal passage of the user to be detected. It will be appreciated that respective passageways may be provided for oral and nasal breathing respectively allowing both of these to be detected independently.

In a further example, the passageway can be connected to a positive airway pressure (PAP) device with allowing air from the PAP device can be delivered to the nose and/or oral cavity of the user via the oral appliance. In this instance, a sensor in the passageway can be used to monitor operation of the PAP device. In particular, this can be used to ascertain a pressure provided by the PAP device ensuring that the PAP device is functioning correctly and providing sufficient airway pressure to the user.

In a further example of the oral appliance, a passageway, opening or channel of the oral appliance or connector system can include flow control element such as a valve, a restrictor or a heat and moisture exchanger. This can be used to moderate, limit or control inflow and/or outflow air from either nasal passageways or the oral cavity. It will be appreciated that multiple different valves could be used for different tidal volumes, and that valves could be adjustable to allow a degree of flow to be controlled. In one example, flow is adjusted manually, but in another example, flow could be controlled based on feedback from the monitoring device, for example to control a relative degree of valve opening based on current breathing. Again, sensors can be used in order to monitor changes in pressure and temperature and thereby assess effectiveness of the control provided.

In one example, the oral appliance includes a number of ports to allow for inhalation or exhalation, wherein at least one port includes a valve to control a flow restriction. This can assist with control of breathing, as well as assisting in generating an elevated air pressure in a passageway and/or airway of a connector system and/or oral appliance, which can assist with monitoring of respiration.

In one particular example, the system includes at least two air pressure sensors for measuring nasal and oral breathing respectively.

Whilst the above described examples have focussed on collection of usage data, which can include sensor data, by assessing whether the device is in use, it will be appreciated that this is not essential, and alternatively sensor data could be collected potentially irrespective of a usage state, to allow respiratory and or sleep characteristics of a user to be measured. In this example, the system would include an appliance monitoring device which in use is attached to or embedded within the oral appliance, the appliance monitoring device including at least one sensor, the at least one sensor including an air pressure sensor that generates a signal indicative of an air pressure, a data store and a processing device. The processing device can receive signals from the at least one sensor; and store sensor data indicative of signals from the sensors in the data store, allowing this to be subsequently transferred to one or more processing systems. The processing systems can then use the sensor data to generate respiratory data indicative of at least one respiratory characteristic of the user of the oral appliance and/or sleep data indicative of at least one sleep characteristic of the user of the oral appliance. It will be appreciated that in one example, this can be used in order to perform a sleep study.

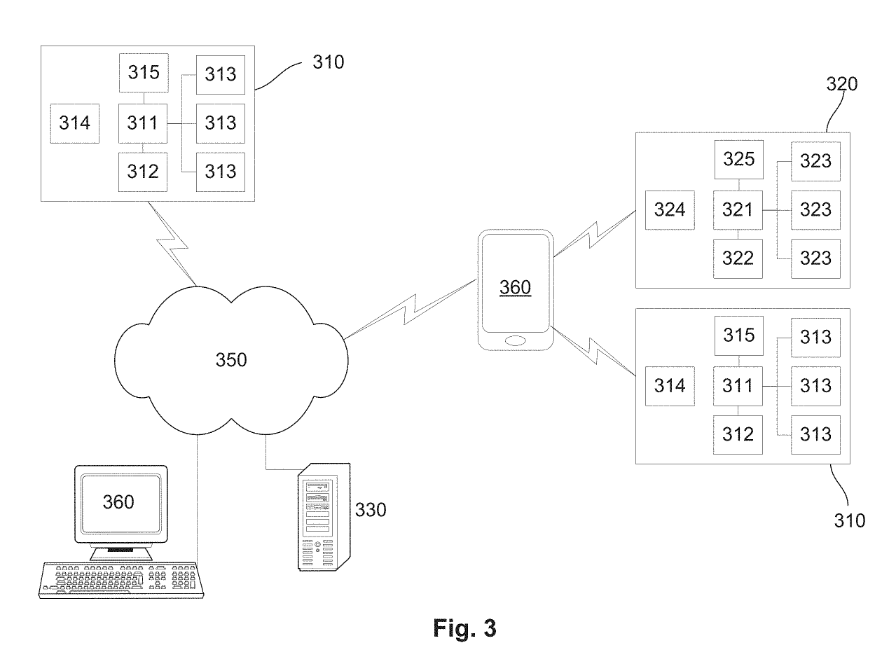

A more specific example will now be described with reference to FIGS. 3 to 6.

In this example, as shown in FIG. 3, the system 300 typically includes a number of monitoring devices 310, each of which is broadly similar to the monitoring device 110 described above, and includes a processor 311, data store 312, sensors 313, a power supply 314 and a transmitter 315.

The system 300 can also include an additional user monitoring device 320, including one or more user sensors for measuring user parameters, such as heart rate, EEG, ECG, pulse ox, or the like. As such monitoring devices are known, these will not be described in any further detail.

Additionally, a number of processing systems including servers 330 and client devices 360 are provided interconnected, via the one or more communications networks 350, such as the Internet, and/or a number of local area networks (LANs), or the like.

Any number of appliance monitoring devices 310, user monitoring devices 320, processing systems could be provided, and the current representation is for the purpose of illustration only. The configuration of the networks 350 is also for the purpose of example only, and in practice the processing systems 330, 360 and appliance monitoring devices 310 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

For example, the appliance monitoring devices 310 could be adapted to communicate with the servers 330 via a communications network, or could communicate directly with a client device 360, with data being transferred from the client device 360 to the server 330, if required. For example, this could be used to allow for cloud based storage of usage or other data, allowing this to be accessed by third parties, such as medical practitioners, or the like. However, it will also be appreciated that the monitoring process described herein could be performed solely by the client devices in which case the servers may not be required.

In one preferred example, the client devices 360 are adapted to communicate with the appliance monitoring devices 310 and retrieve sensor data therefrom, performing analysis of this and generating representations for the user. Additionally, the client devices 360 provide recorded data to the servers 330, allowing additional analysis to be performed and to allow access and review by third parties such as medical practitioners.

Whilst the servers 330 are shown as single entities, it will be appreciated they could include a number of processing systems distributed over a number of geographically separate locations, for example as part of a cloud based environment. Thus, the above described arrangements are not essential and other suitable configurations could be used.

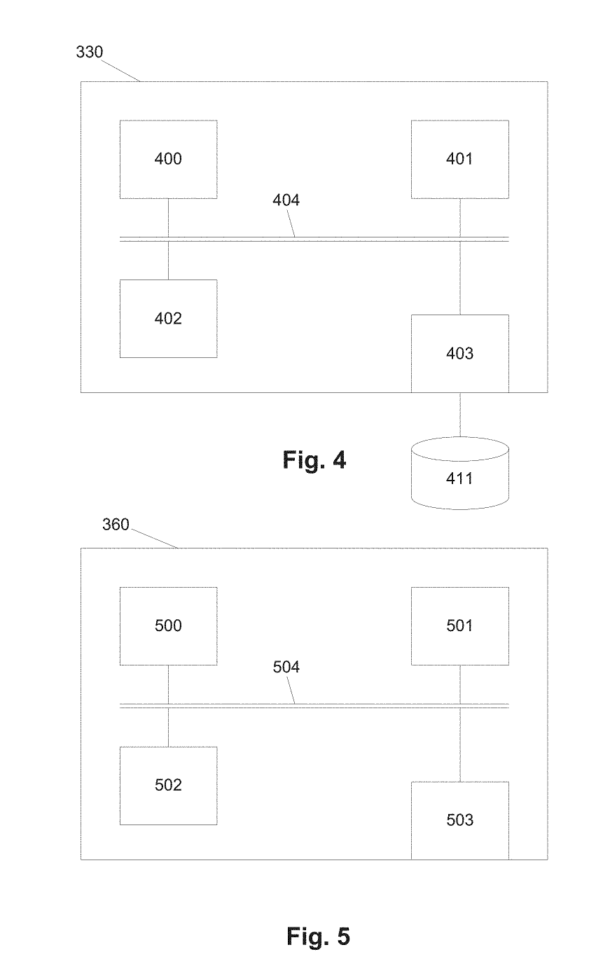

An example of a suitable server 330 is shown in FIG. 4. In this example, the server 330 includes at least one microprocessor 400, a memory 401, an optional input/output device 402, such as a keyboard and/or display, and an external interface 403, interconnected via a bus 404 as shown. In this example the external interface 403 can be utilised for connecting the server 330 to peripheral devices, such as the communications networks 350, databases 411, other storage devices, or the like. Although a single external interface 403 is shown, this is for the purpose of example only, and in practice multiple interfaces using various methods (eg. Ethernet, serial, USB, wireless or the like) may be provided.

In use, the microprocessor 400 executes instructions in the form of applications software stored in the memory 401 to allow the required processes to be performed. The applications software may include one or more software modules, and may be executed in a suitable execution environment, such as an operating system environment, or the like.

Accordingly, it will be appreciated that the server 330 may be formed from any suitable processing system, such as a suitably programmed PC, web server, network server, or the like. In one particular example, the server 330 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

As shown in FIG. 5, in one example, the client device 360 includes at least one microprocessor 500, a memory 501, an input/output device 502, such as a keyboard and/or display, and an external interface 503, interconnected via a bus 504 as shown. In this example the external interface 503 can be utilised for connecting the client device 360 to peripheral devices, such as the communications networks 350, databases, other storage devices, or the like. Although a single external interface 503 is shown, this is for the purpose of example only, and in practice multiple interfaces using various methods (eg. Ethernet, serial, USB, wireless or the like) may be provided.

In use, the microprocessor 500 executes instructions in the form of applications software stored in the memory 501, to allow communication with the servers 330 and/or monitoring devices 310.

Accordingly, it will be appreciated that the client device 360 be formed from any suitably programmed processing system and could include suitably programmed PCs, Internet terminal, lap-top, or hand-held PC, a tablet, a smart phone, or the like. However, it will also be understood that the client device 360 can be any electronic processing device such as a microprocessor, microchip processor, logic gate configuration, firmware optionally associated with implementing logic such as an FPGA (Field Programmable Gate Array), or any other electronic device, system or arrangement.

Example processes performed by the system will be described in further detail below. For the purpose of these examples it is assumed that the servers 330 typically execute processing device software, allowing relevant actions to be performed, with actions performed by the server 330 being performed by the processor 400 in accordance with instructions stored as applications software in the memory 401 and/or input commands received from a user via the I/O device 402. It will also be assumed that actions performed by the client devices 360, are performed by the processor 500 in accordance with instructions stored as applications software in the memory 501 and/or input commands received from a user via the I/O device 502.

However, it will be appreciated that the above described configuration assumed for the purpose of the following examples is not essential, and numerous other configurations may be used. It will also be appreciated that the partitioning of functionality between the different processing systems may vary, depending on the particular implementation.

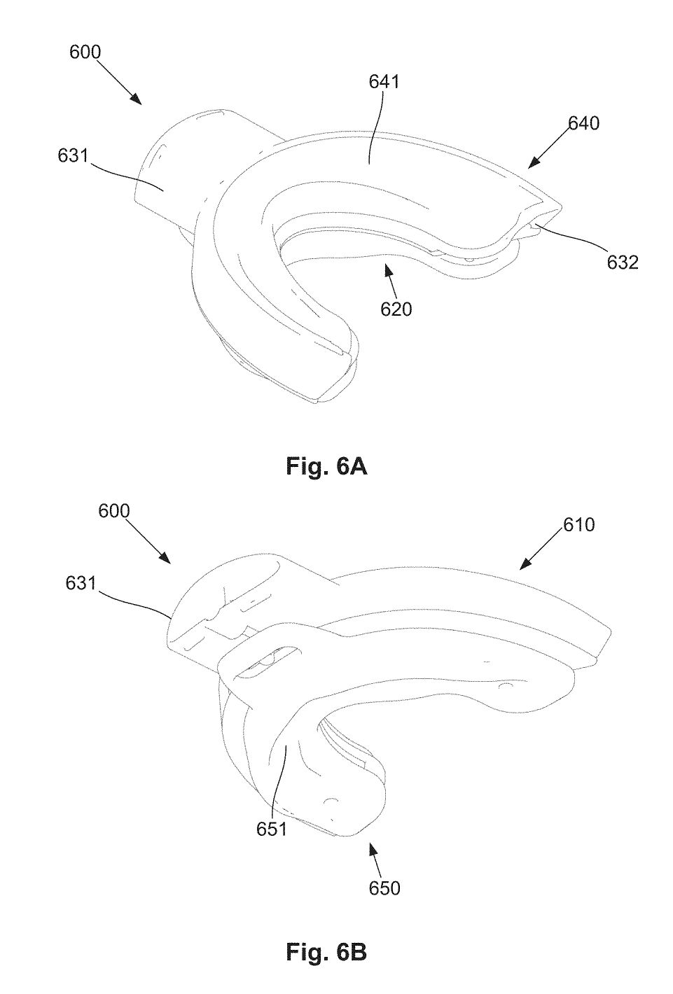

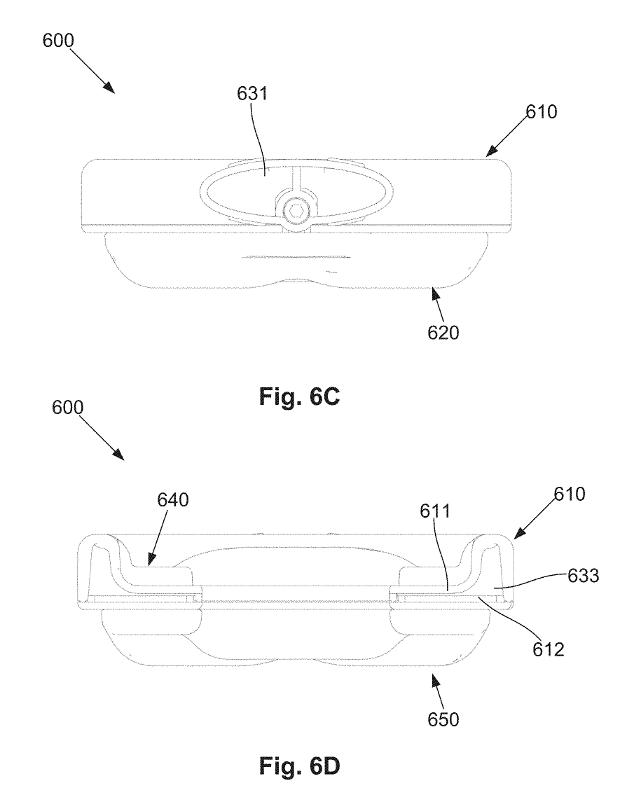

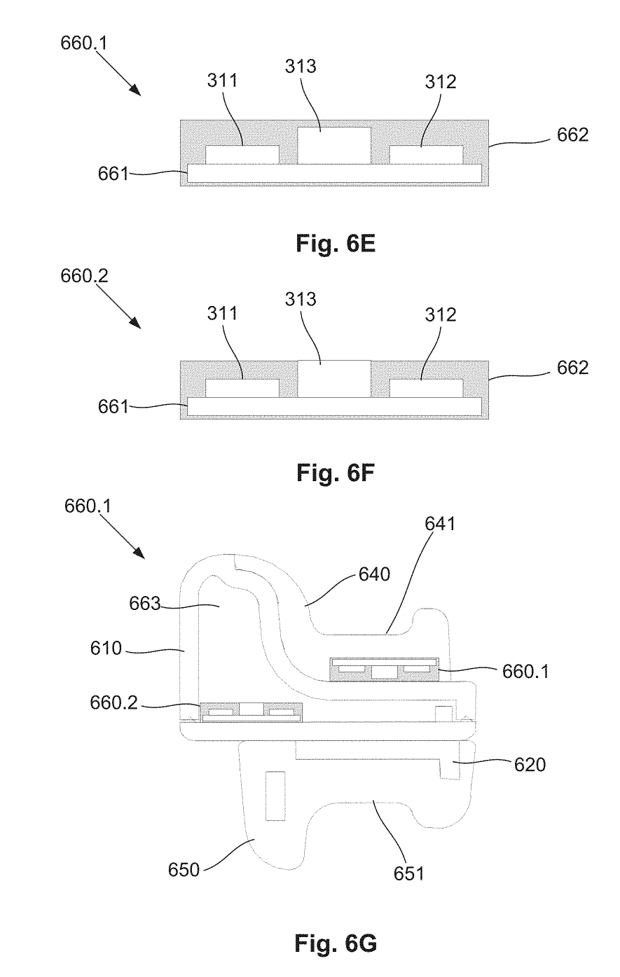

An example of an appliance for providing breathing assistance and an associated appliance monitoring device will now be described with reference to FIGS. 6A to 6G.

In this example, the apparatus 600 includes a first body 610 and a second body 620. The first body 610 includes a first opening 631 extending forwardly from a front of the body to define an extra-oral opening, for allowing airflow between the lips of the user, and at least one second opening 632 provided in the oral cavity of the user, the first and second openings being interconnected via one or more channels 633 to allow airflow into and out of a posterior region of the oral cavity.

Each of the first and second bodies is coupled to a respective bite member 640, 650, which includes a respective recess 641, 651 moulded to fit the user's teeth. The term bite member will be understood to include any form of member that is shaped to conform to, or otherwise suitably receive, the user's teeth. In some instances the bite member is referred to as an “insert”, and the terms should be considered as being interchangeable. This arrangement allows the apparatus to be positioned in and retained within the user's mouth in use.

Thus, the device has a first opening 631, defined by a tubular body, which can extend beyond the lips, or at least keep the lips apart, to allow airflow therethrough. Air passes through airways defined by the channels 633, and is directed into a posterior region of the mouth through second openings 632, bypassing issues with tongue and lower jaw position. In more severe cases, the first opening 631 can be connected to an external device, such as a CPAP (Continuous Positive Airway Pressure) machine, air supply, or the like providing more comfort and increased patient compliance compared to a mask. In such situations, the channels 633 may be divided into two or more airways working together or in opposite directions.

Providing air flow directly into a posterior portion of the user's oral cavity has a number of benefits. In particular, this avoids obstructions created by the nasal cavity, soft palate and tongue which can lead to snoring and apnoea events, and helps reduce the drying effects of air flow, which can in turn lead to user discomfort. This makes the apparatus comfortable to wear whilst ensuring an unobstructed air flow thereby preventing snoring and apnoea events. Thus, for example, nasal obstructions can be bypassed by air flow through the apparatus, thereby bypassing the nasal airway or adding to it in the case of a partial obstruction. Furthermore, air flowing below or on both sides of the soft palette helps prevent collapse of the soft palate, which can in turn lead to additional obstruction.

In one example, the first body 610 is made of a base 612 and a cover 611, which cooperate to define the channel 633. The base 612 can be a substantially planar base, optionally including ridges or guides to facilitate attachment to the cover, whilst the cover 611 is shaped to define the channels, the openings and to allow attachment of the bite member. The base and cover can be connected via any appropriate mechanism, such as a mechanical coupling, a friction fit, interference fit, adhesive, ultrasonic welding or the like.

In one example, the bodies are manufactured using injection moulding, and in particular by injection moulding a polymer, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials. The formation of the first body from the base and cap facilitates the process of injection moulding the body ensuring that the channels can be formed, which would be an otherwise difficult process.

However, it will be appreciated that this arrangement is not essential and alternatively the first body 620 could be formed as an integral body, for example using an additive manufacturing technique, such as 3D printing or the like.

The bodies can be coated with a medical grade polymer and in one example, a medical grade elastomer, such as silicone or polyurethane, epoxy or parylene, for improved comfort as well as ensuring biocompatability.

Typically one or more standard sizes of first and second body can be produced, with an appropriate first and second body being selected based on a closest fit to the intended user. Custom fitting can then be achieved using customised bite members positioned between the user's teeth and the respective body in use. Each bite member is typically customised for a user's teeth and is adapted to be removable and/or replaceable. Additionally, the first and second bodies can be resized, for example by cutting or otherwise removing part of the body, such as tabs provided on a rearward part of the first body.

Bite members can also be made by injection moulding similar materials to the bodies, by additive manufacturing, such as 3D printing, and/or could be manufactured by having the user bite into a material that can be moulded to the shape of the user's teeth and then solidified. For example, this could include UV curing, using a thermosetting material or the like. In one example, the bite members are formed from boil and bite materials, such as Ethylene-vinyl acetate or the like, although silicone or other materials, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials, could be used. For example, this could be used to allow a user to mould the bite members at home by biting into a bite into a member made of a suitable material such as silicone.

In one example, the apparatus can be used be with a plurality of different bite members, which can be used for example to provide different levels of fit, comfort, support or the like. The bite members can also be either a temporary or semi-permanent, and may be made from different materials depending on their intended use. For example, a temporary bite member could be created upon the initial fitting of a breathing assist apparatus, using a silicone which is moulded in-situ, with this being replaced by a subsequent semi-permanent bite member, such as an injection moulded bite member, once there has been opportunity for this to be manufactured. This allows an initial fitting to be performed when the apparatus is initially supplied with temporary bite members, with semi-permanent acrylic bite members being subsequently manufactured and provided to the user once ready.

Whilst the bite members could be fitted using any suitable technique, in one example, the bite members can be attached to the first and second bodies using adhesives, mechanical couplings, such interference fits, or the like.

The use of bite members, as described above, allows a variation in the shape of teeth and jaws that can be accommodated by the first and second bodies. This allows the majority of individuals to be fitted by selecting one of a number of defined template bodies, having standard sizes/dimensions.

Additionally, bite members can be manufactured so as to be thermformable, allowing these to be reshaped slightly by heating to accommodate changes in the user's jaw positioning or shape over time. Even such semi-permanent bite members would typically undergo wear and potential discolouration, and hence can be replaced periodically. Despite this, the first and second bodies can be reused as needed, so bite members can be recreated from the previously scanned moulds. The ability to remove bite members allows these to replace and/or cleaned and re-used as required. Similarly, the bodies can also be cleaned and/or sterilised prior to re-use.

When an airway is provided, the channels can have a wide variety of configurations and may be sized and shaped depending on the anatomy of the oral cavity of the user. This is typically done to maximise the available airway, whilst ensuring comfort for the user. In one example, the total airway has a cross sectional area of at least one of 50 mm² to 70 mm², 70 mm² to 90 mm², 90 mm² to 100 mm², 100 mm² to 110 mm² and preferably 70 mm² to 90 mm², at least 50 mm², at least 70 mm², at least 90 mm², at least 100 mm² or at least 110 mm². The dimensions selected will vary depending on a wide range of factors, including whether the device is required to provide a partial or complete airway, for example to bypass a partial or complete blockage. Additionally, this will depend on the intended use and the associated airflow requirements. Typically the dimensions of the channels and/or openings 631, 632 are selected so that in conjunction with the user's existing airways, the total airway available corresponds to the cross sectional area of an airway in a healthy subject for both nasal and pharyngeal airways. In any event, the cross sectional areas used will depend on the preferred implementation and intended use, so for example, a smaller cross sectional area may be used for children, adolescents, or individuals with only partial obstructions. In contrast increased cross sectional areas may be used where a high flow rate is required, for example in the event that the device is to be used to provide breathing assistance during exercise.

In one example, each channel includes a portion extending through the buccal cavity, between the user's cheeks and teeth, and a portion extending between the user's maxillary and mandibular teeth. This arrangement maximises the cross sectional area of the channels, whilst maintaining comfort for the user, by distributing the airway between the user's teeth and cheeks and between the user's teeth.

The cross sectional area of the first and second channel portions can vary between the first and second openings, allowing the overall cross section of the channel to be maintained, whilst having the channel conform as far as possible to the natural space available in the oral cavity. It will be appreciated that any variation can be used, depending for example on the configuration of the user's oral cavity.

In one example, the second openings are angled inwardly at between 10° and 50°, more typically between 20° and 40° and preferably about 30° to assist in airflow into and out of the oral cavity, and in particular to direct airflow towards the centre of the oral cavity. Additionally and/or alternatively, the second openings are positioned over the last or back tooth on each side of the top jaw. As previously mentioned, the provision of an airway, although advantageous is not essential.

In one example, the apparatus further includes an adjustable mounting configured to interconnect the first and second bodies to allow a relative position of the first and second bodies to be adjusted. The form of the adjustable mounting will vary depending upon the preferred implementation and specific examples will be described in more detail below.

Thus, the apparatus provides an oral appliance for providing breathing assistance. This can be used during sleep, for example for the treatment of both, snoring and sleep apnoea, and can also be used at other times, for example in the treatment of respiratory conditions, such as emphysema, and to assist in jaw placement to provide an adequate airway, for use during surgery, CPR (Cardiopulmonary resuscitation), or the like.

As respective sets of teeth of the user are provided in the bite members coupled to the first and second bodies, adjusting the relative position of the first and second bodies can be used to selectively position the mandibular teeth relative to the user's auxiliary teeth, for example to provide mandibular advancement. In this regard, it is known that mandibular advancement can assist in holding open the user's airway, which in turn can reduce snoring. For example, temporomandibular joint disorder (TMD) arises when the upper and lower jaws are misaligned. This may be naturally occurring or can result from injury, or the like. Regardless, such jaw misalignment tends to contribute to airway obstructions by changing the shape of the upper airway, and moving the tongue towards the posterior of the oral cavity, which can in turn exacerbate issues associated with OSA and snoring. Accordingly, by allowing the relative position of the first and second bodies to be adjusted, this allows the jaws of the user to be aligned thereby reducing the effects of TMD, and hence further reducing the likelihood of snoring and OSA.

Accordingly, the provision of the adjustable mounting, allowing the relative position of the first and second bodies to be controllably adjusted, in turn allows a relative position of the lower jaw to be adjusted, for example to provide for mandibular advancement. This can assist significantly in the reduction of snoring.

In one example, the apparatus allows a relative position of the first and second bodies to be adjusted in a longitudinal direction, with the second body being positionable at points between rearward and forward positions respectively. In this regard the longitudinal direction is typically aligned with a dental midline of the user in use, so that this configuration can provide mandibular advancement, although it will be appreciated that other forms of adjustment could be provided.

The adjustable mounting is typically configured to further allow relative lateral movement of the first and second bodies, and to allow relative to-and-fro movement of the first and second bodies. These movements can be largely unconstrained, allowing a user to move their jaw laterally, or to open and close their jaws, whilst remaining engaged with the apparatus. This can aid comfort when using the device, whilst ensuring mandibular advancement is maintained.

The adjustable mounting can be of any appropriate form. In one example, this includes a threaded shaft rotatably mounted to the first body, arranged to extend in a longitudinal direction and which has a threaded carriage mounted thereon to allow the carriage to move longitudinally in response to rotation of the shaft. The carriage supports a peg, which in use engages a slot in the second body, allowing the second body to move longitudinally as the shaft is rotated.

In one example, the slot is a keyhole slot, having a wide end defining an eyelet into which the peg can be bite membered, and a narrow section for retaining the peg in place. The slot can have a slot depth greater than a head height of the peg, and wherein the lip has a lip height smaller than a shaft height of the shaft to allow the peg to move in the slot to allow for relative to and fro movement of the first and second bodies.

In one example, the threaded shaft is mounted at least partially within the first opening in the first body. To achieve this, the threaded shaft can include a plug that is rotatably mounted within a corresponding socket in the first body, whilst the threaded shaft includes a head at a second end, which allows the shaft to be rotated, for example through engagement with a tool such as a hex key or similar. The first body typically also includes a collar extending at least partially around the threaded shaft approximate the second end with the head being recessed within the collar, to thereby hold the second end in position, whilst allowing rotation of the head end.

Accordingly, it will be appreciated that the combination of the airway and the ability to adjust the relative position of the first and second bodies can provide assistance beyond that afforded by either the airway or mandibular advancement alone. However, it will also be appreciated from examples described in more detail below that the airway is not necessarily required depending on the intended usage of the arrangement. In this regard, a similar configuration of adjustable bodies can be used for the purpose of adjusting a relative position of the use's jaws, for example to correct muscular-skeleto defects and/or for use in creating moulded bite members for use with an oral appliance, as will be described in more detail below.

It will be appreciated that the overall configuration of the apparatus described above, when the first and second bodies are combined, is similar to arrangements described in copending applications PCT/AU2012/000565 and PCT/AU2015/050144, the contents of which are incorporated herein by reference.

For example, the apparatus can include a filter for filtering air flowing through the apparatus. This can help remove particulates, pollen or other contaminants entrained in air flowing into the device, which can assist in reducing respiratory irritation, which can in turn exacerbate snoring and breathing difficulties. The filter can be positioned anywhere within the body 610, but is typically provided within the first opening 631, thereby allowing this to be easily removed and replaced if required. The filter could be of any suitable form and could include a porous plastic or cloth based filter, and may include additional materials for added functionality. For example, the filter can also include activated carbon for filtering out pollution/bacteria.

Additionally, and/or alternatively a heat/moisture exchanger can be provided that controls the water and temperature content of the air being inhaled by exchanging heat and moisture with exhaled air. Examples of such exchangers can be found for example in U.S. Pat. No. 5,433,192, and these will not therefore be described in any further detail.

Additionally or alternatively, the apparatus can include a valve (not shown) for regulating air flow through the apparatus. In one example, this can be used to resist outflow of air from the second openings to the first opening. This can assist in regulating breathing and in particular allow for rapid inhalation, whilst ensuring slower exhalation, thereby optimising gas exchange within the lungs, for example to minimise the chances of hyperventilation. The valve can be of any suitable form, such as a ball valve, umbrella valve, or the like, and can be adjustable or titratable to ensure that the level of flow control is appropriate to the user. Such adjustment could be performed manually, or could be performed automatically based on the user's current breathing state.

An example of the physical configuration of the appliance monitoring device is shown in FIGS. 6E and 6F.

In each example, the arrangement typically includes a circuit board 661 having components mounted thereon including for example the processing device 311, memory 312 and one or more sensors such as a pressure sensor 313. In one particular example, the pressure sensor 313 is a MEMS (microelectromechanical) nano pressure sensor, and in particular an ultra-compact piezoresistive absolute pressure sensor having a sensing element and integrated circuit interface allowing this to be mounted directly to the circuit board. The board and associated componentry including the pressure sensor is encased in an epoxy resin 662 or other similar material.

In the example of FIG. 6E, the pressure sensor 313 is entirely enclosed within the epoxy resin, meaning the pressure sensor detects physical pressure on the epoxy resin and therefore act as a contact sensor. In contrast, in the arrangement shown in FIG. 6F, the upper surface of the pressure sensor extends beyond the epoxy resin and hence is exposed to the atmosphere allowing this to function as an air pressure sensor.

The pressure sensors may be provided in a variety of locations within the oral appliance. In the example of FIG. 6G a cross-sectional view through the channel is shown. In this example, an appliance monitor device 660.1 including a contact sensor is embedded within the bite member 640 so that contact between the bite member and body 610 can be sensed. This can be used to detect pressure of a user's teeth on the bite member in turn indicating that the oral appliance is in use, as well as allowing a contact pressure to be detected, which can in turn be indicative of bruxism, or the like. Additionally, in this example an appliance monitor device 660.1 including a pressure sensor functioning as an air pressure sensor is mounted in the channel 632 allowing changes in air pressure within the channel to be sensed.

Accordingly, it will be appreciated that in the above described arrangement, suitable configuration of multiple appliance monitoring devices can be used in order to allow different aspects of use, and in particular, contact and air pressure, to be monitored. Whilst the above described arrangement uses two separate monitoring devices, this is for the purpose of ease of illustration only and it will be appreciated that in practice, multiple sensors could be incorporated into a single appliance monitoring device. The sensors could also be provided in their own respective housing, and connected to a single central processing device 311 via suitable wiring, allowing the sensors to be distributed throughout the oral appliance.

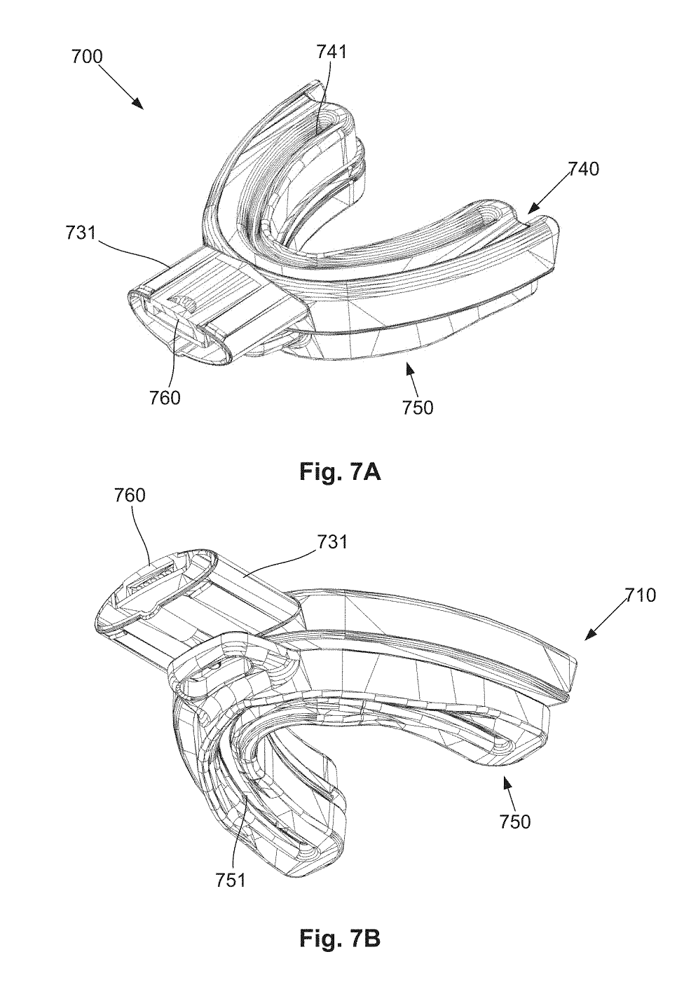

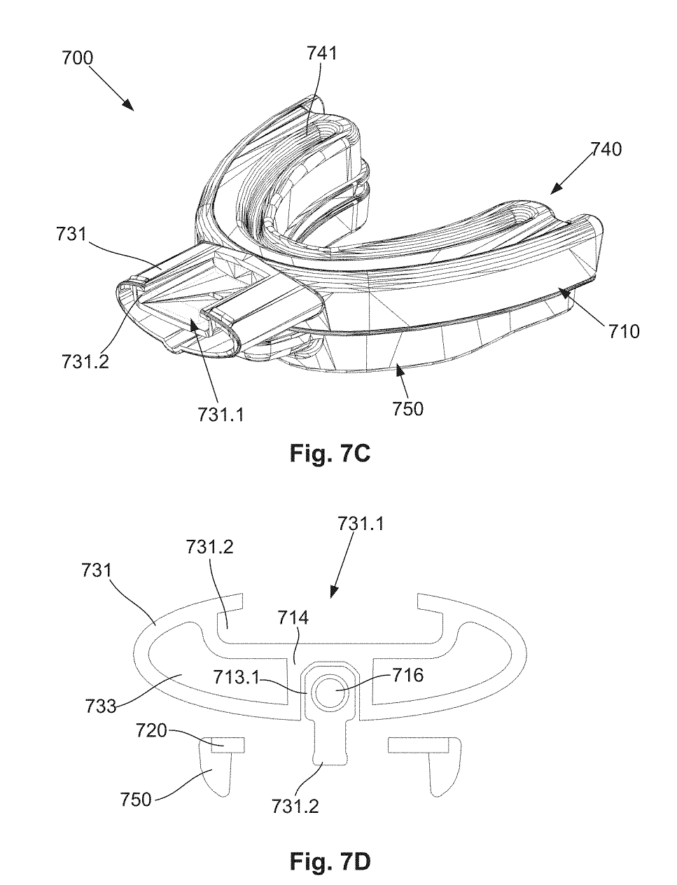

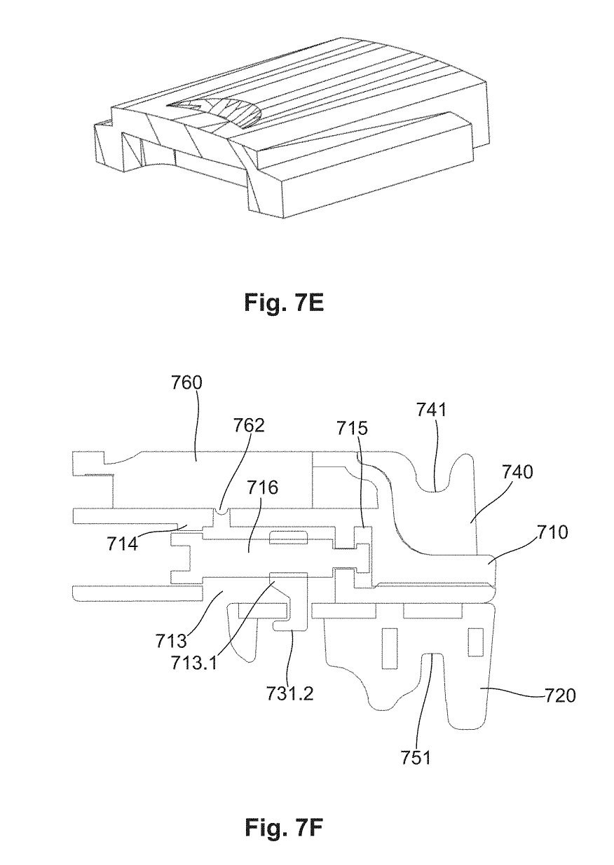

A further example arrangement will now be described with reference to FIGS. 7A to 7F, which shows a modified version of the oral appliance of FIGS. 6A to 6D. Similar features are therefore denoted by similar reference numerals increased by 100 and these will not therefore be described in further detail.

In this example, the extra-oral opening 731 is provided by a tubular body including a slot 731.1 in an upper surface, with mounting grooves 731.2 extending along each side of the slot. In this example, the appliance monitoring device 760 is shaped to engage the mounting grooves 731.2 and fit within the slot 731.1, so that an upper surface of the appliance monitoring device 760 is contiguous with the upper surface of the opening 731. A lower surface of the appliance monitoring device 760 can include a ridge 762 or other projection that engages with a corresponding opening in a base of the slot to thereby lock the appliance monitoring device 760 into a fixed position in the slot 731.2. The appliance monitoring device 760 therefore forms an integral part of the opening 760. In this example, the appliance monitoring device 760 can be fitted with a contact pressure sensor in an upper surface, and air pressure and/or temperature sensors in a lower surface, so that the appliance monitoring device 760 can monitor both contact of the user's lips with the upper surface of the opening 731, as well as air and/or temperatures within the opening, and in particular for air flow through the opening.

The opening 731 includes a collar 714 mounted on an inner underside of the opening 731. A socket 715 is provided in the first body 710 within an outer side wall, in line with the collar at the back of the opening 731, which received a threaded shaft 716. The lower side of the collar 714 and opening 731 define a carriage slot 713 extending from the collar to the socket opening, allowing a carriage 713.1 to be mounted on the threaded shaft so that this can be moved longitudinally along the shaft in response to rotation of the shaft. A peg 713.2 is mounted on the carriage, and engages a keyhole slot 723 in the second body 720 and bite member 750, allowing the second body 720 to be adjustably mounted to the first body.

However, it will be appreciated that this is not essential, and in another example, the first and second bodies could be provided in a fixed static arrangement.

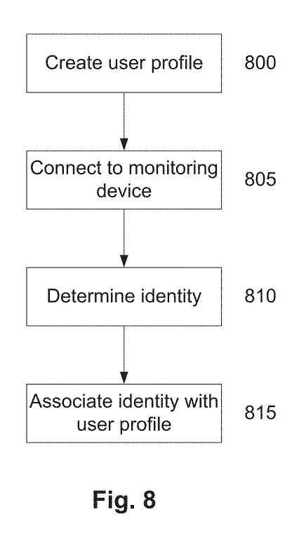

Example processes for monitoring usage of the oral appliance, using the system described above with respect to FIG. 3, will now be described in more detail. Before the system is initially used, the compliance monitoring device is typically associated with a user, and a user profile created, and an example of this process will now be described with reference to FIG. 8.

In this example, at step 800 the user profile is created. This is typically performed using a processing device, such as a client device 360, for example by having the user load an app on their client device 360, and then enter any required information. This process will typically involve determining information regarding user, such as information regarding physical characteristics, such as age, height, weight or the like, as well as details of particular conditions of interest to be monitored, such as sleep apnoea, snoring or the like. The information can be collected in any appropriate manner, such as asking a series of questions, having the user select options from a drop-down box or the like. It will be appreciated that this process may be performed in conjunction with a medical practitioner, such as a sleep therapist, and that some of the information could be imported from a remote data source, such as an electronic medical record or the like.

As part of the process, the user may also be required to define compliance requirements, and optionally configure alerts, for example to notify the user or another individual if compliance requirements are not met, as will be described in more detail below. Additionally, permissions to allow third parties access to any collected data may also need to be defined.

At step 805, the user is typically required to pair their compliance monitoring device, with a suitable processing system, such as the client device 360, allowing usage data to be downloaded thereto. In one example, this is achieved by pairing the appliance monitoring device with a smart phone, using Bluetooth or another suitable technology.

At step 810, the processing system determines an identifier associated with the compliance monitoring device, and records this as part of the user profile at step 815. This ensures that usage data collected from the appliance monitoring device is correctly associated with the respective user moving forward.

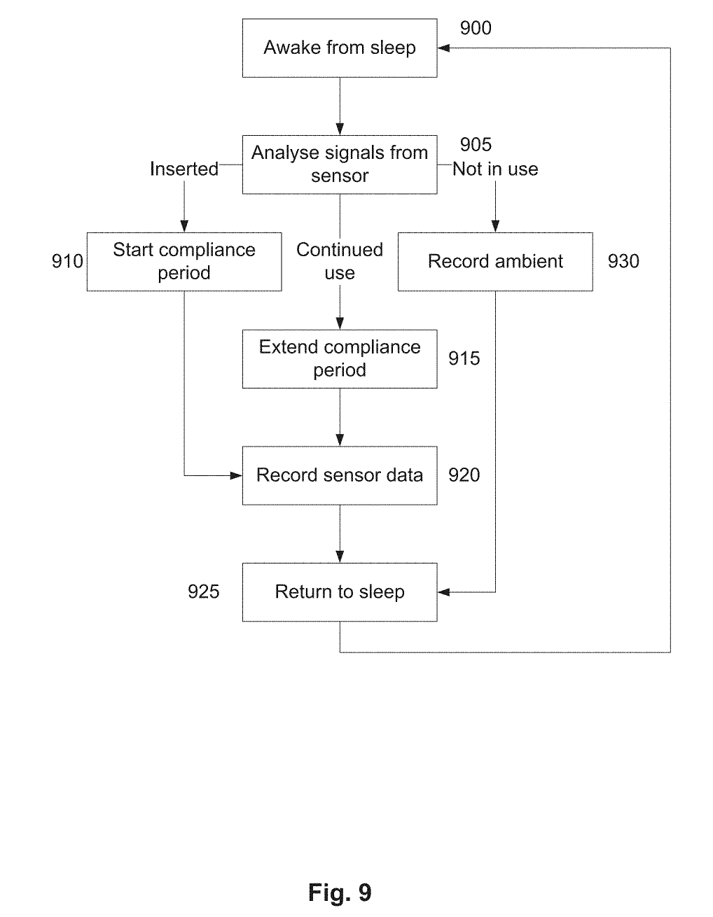

Once the system has been configured, it is then possible to perform monitoring and an example of this process will now be described with reference to FIG. 9.

In this example, the processing device and associated electronics are adapted to enter a low power “sleep” mode when sensing is not being performed. This is utilised in order to conserve battery life and hence increase the amount of time during which monitoring can be performed. The monitoring device is typically adapted to wake from sleep after a predetermined time at step 900 and/or upon detection of a touch event.

At step 905 the processing device 311 analyses signals from one of the sensors 313 and uses this to determine if the oral appliance is currently in the process of being inserted, is in continued use, or is not in use. In the event that the device is being inserted, the processing device starts a compliance period by creating associated compliance data at step 910 and then optionally commences recording sensor data at step 920. Sensor data is recorded for a defined length of time, such as a few seconds, to accumulate data regarding respiration of the user and/or operation of a connected PAP device. In this regard, it will be appreciated that sensor data need not be recorded if compliance sensing only is being performed. Following this the appliance monitoring device returns to sleep at step 925 for a defined period of time, before the cycle is repeated.

In the event that the processing device determines that a compliance period has already commenced on a previous cycle, and that the device is still in use, the processing device 311 operates to extend the compliance period by updating the compliance period at step 910, and optionally records further sensor data at step 920. Again this is performed for a set length of time before the device returns to sleep at step 925.

Alternatively, if it is determined that the device is not in use, then data from the sensors 313, and in particular from an air pressure and/or temperature sensor, is used to record ambient air pressures and/or temperatures at step 930. These values can be utilised in evaluating sensor data collected during use of the device as will be described in more detail below. Again, once ambient data is recorded the monitoring device returns to sleep at step 925.

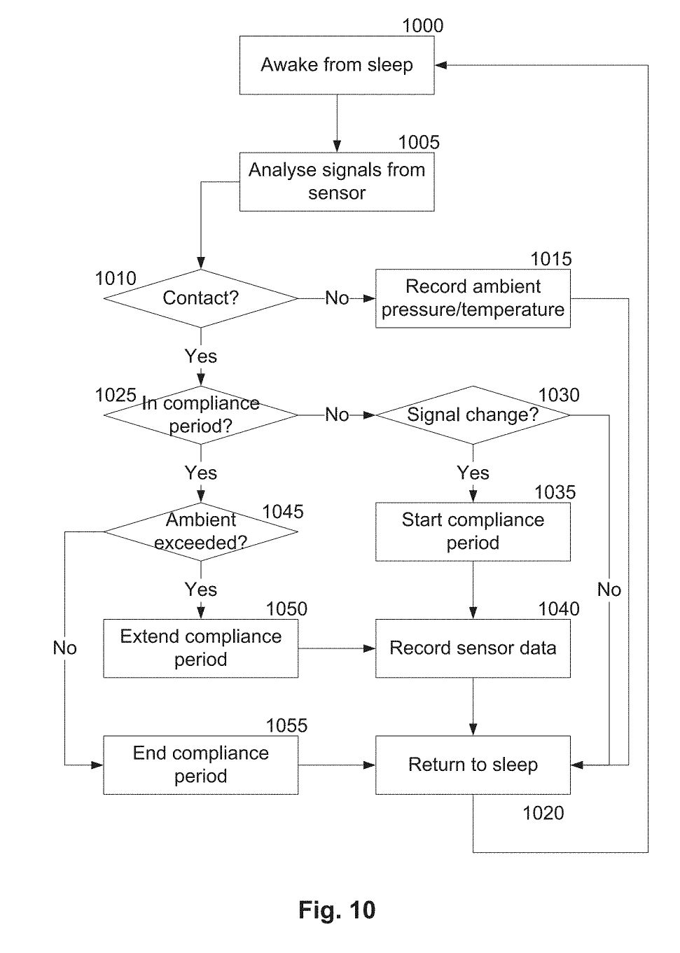

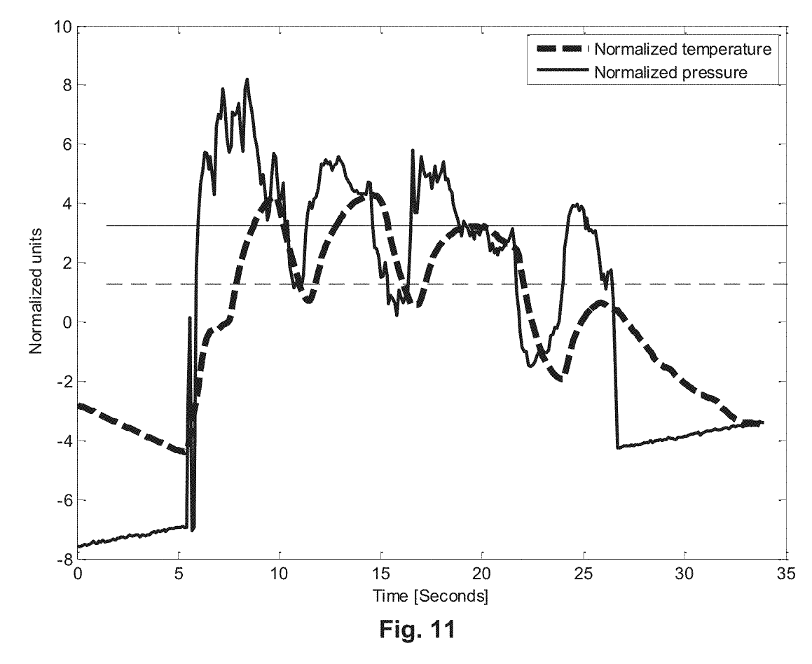

Accordingly, it will be appreciated that the above described process allows the monitoring device to periodically assess whether the oral appliance is in use, and if so capture data regarding that use, whilst also simultaneously determining a compliance period, corresponding to a time interval during which the monitoring device is being used. The manner in which use of the device is assessed will vary depending on the preferred implementation and can be based on detected contact with a user as well as other signals from the sensors, and a specific example of this will now be described in more detail with reference to FIG. 10.

In this example, the monitoring device awakes from sleep at step 1000 with the processing device operating to analyse signals from the sensors at step 1005. In particular, the processing device will analyse signals from a contact sensor to determine if a user is in contact with the device at step 1010. If no contact is detected, ambient temperature and pressures are recorded at step 1015 with the device returning to sleep at step 1020.

Alternatively if contact is detected, the processing device determines if a compliance period has already commenced on a previous cycle. If a compliance period has not commenced, at step 1030 the processing device monitors air pressure and temperature to identify a significant change in pressure or temperature. In this regard, a significant change in temperature or pressure can be used to indicate that the device has been inserted into the user's mouth. An example of this is shown in FIG. 11, which shows the typical air pressures and temperatures within the extra-oral appliance, prior to use (0-5 seconds), upon insertion into the user's mouth (5 seconds), during a period of use (5-26 seconds), and following use (26-35 seconds).

Upon detection of an increase in air pressure and/or temperature, the processing system 311 commences a compliance period at step 1035, logging an indication of the commencement time and date, before recording air pressure and temperature sensor data at step 1040. Otherwise, if there is no signal change, or once sensor data has been recorded the device returns to sleep state at step 1020.

In the event that it is determined that a compliance period has already commenced at step 1025, the processing device 311 determines if the air pressure and temperature exceed the ambient pressure and temperature. Assuming this to be the case, this confirms that the device is in use and the compliance period is extended at step 1050 with sensor data being recorded at step 1040. Otherwise, if the ambient pressure and temperature are not exceeded the compliance period is ended at step 1055.

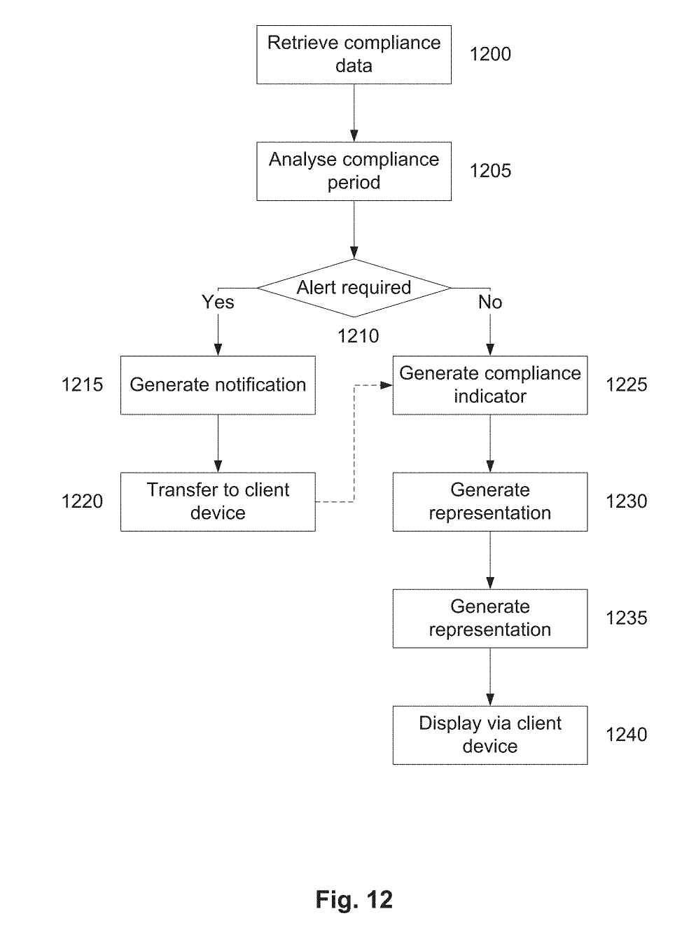

Accordingly, it will be appreciated that the above described approach uses a combination of contact sensing and sensing of air pressure and temperature in order to determine an appliance usage status. This information is then used to perform compliance monitoring, as well as allowing sensor data to be recorded for further analysis, an example of which will now be described with reference to FIG. 12.

In this example, compliance data indicative of a compliance period is retrieved by a processing system, such as a server 310 or client device 360 at step 1200. The compliance data will typically include information regarding a compliance period start time and date, as well as a duration of the compliance period. The compliance data will also typically include an identifier indicative of the appliance monitoring device used to record the compliance data, allowing the processing system 310 to retrieve the user profile of the respective user.

At step 1205 compliance periods are analysed to determine if an alert is required. In this regard, alerts can be set as part of the user profile to notify the user and/or a medical practitioner or other individual, if the user is failing to meet compliance requirements. If it is determined that an alert is required at step 1210, a notification can be generated at step 1215 with this being transferred to a client device 360 at step 1220.

Alternatively, or following this, a compliance indicator can be generated at step 1225, which is indicative of the measured compliance periods, and in particular whether these meet the necessary compliance periods. This information can then be stored as part of the user profile at step 1230, allowing this to be subsequently retrieved as required. In this regard, the compliance indicator can be used to generate a representation at step 1235, which can then be displayed to the user and/or another third party, via a client device at step 1240. The representation could be of any appropriate form, and could be a simple indication that compliance requirements are or are not met, or alternatively could include more detailed information, such as a time, date and duration of each compliance period. The indication can be displayed in any suitable manner, including in a textual, numerical or graphical form.

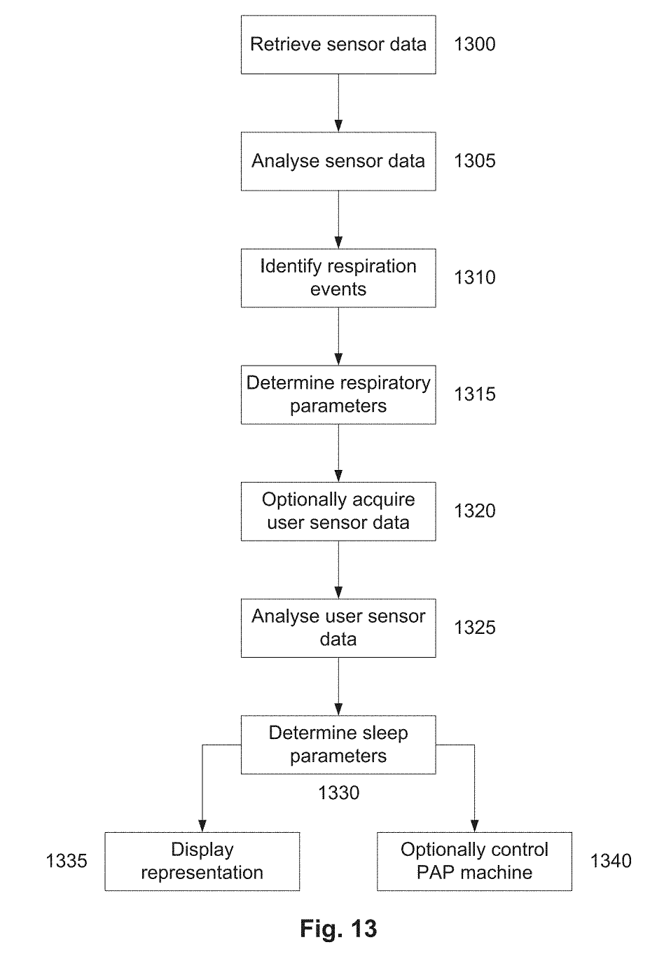

An example of the process for analysing respiration and/or sleep data will now be described with reference to FIG. 13.

In this example, sensor data is retrieved from the monitoring device at step 1300. The sensor data is analysed at step 1305 in order allow respiratory events to be identified at step 1310. In particular, this can involve identifying changes in pressure and/or temperature similar to those shown in FIG. 11, which can be indicative of changes in air flow and hence inhalation or expiration events. This information is used to determine respiratory data, such as a breathing rate, breathing magnitude or the like. Additionally, frequency analysis, such as performing a Fourier transform can be performed in order to determine and analyse frequency components of breathing, which can in turn allow snoring to be identified.

Additionally, user sensor data can be obtained from a user monitoring devices 320 at step 1320, allowing the user sensor data to be analysed in conjunction with the sensor data from the appliance monitoring device, and used in determining sleep parameters at step 1330. In this regard, information regarding breathing can be combined with information regarding heart rate, pulse oximetry data, or the like, allowing a variety of sleep parameters to be ascertained.

Information regarding the respiratory and/or sleep parameters can be associated with the user profile and displayed as part of a representation at step 1335. The representation could again be of any appropriate form, and could be a simple indication that compliance requirements are or are not met, or alternatively could include more detailed information, such as a time, date and duration of each compliance period. The indication can be displayed in any suitable manner, including in a textual, numeric or graphical form.

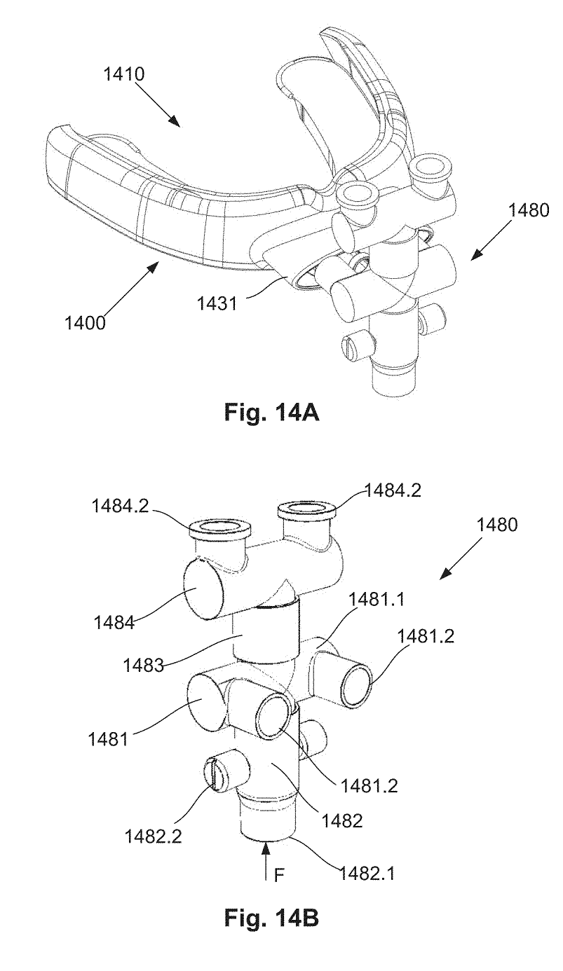

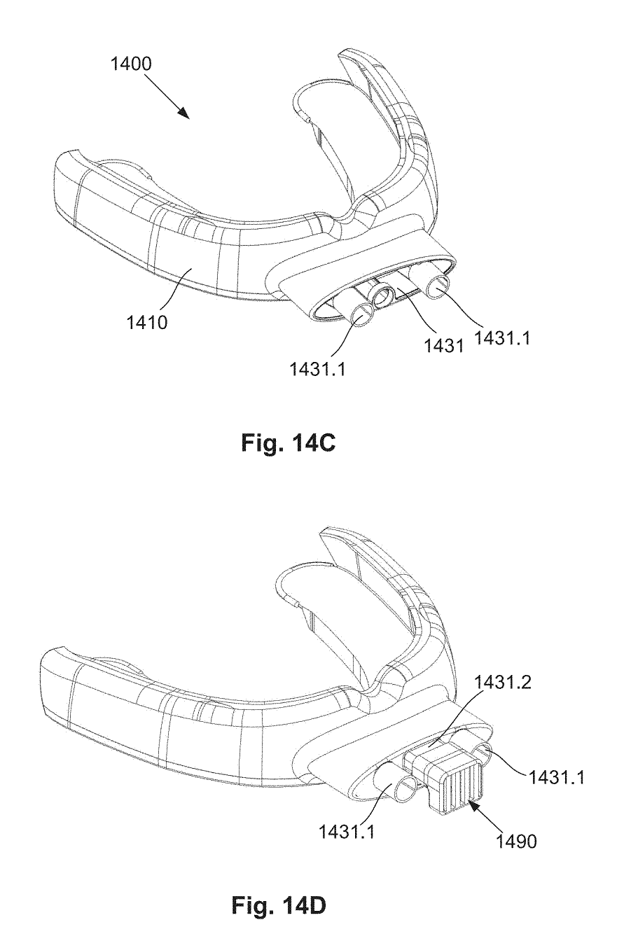

As previously mentioned, the system can also be adapted to monitor operation of a PAP device connected to an oral appliance and an example of a system for providing breathing assistance to a user will now be described with reference to FIGS. 14A to 14D.

In this example, the system includes an oral appliance 1400 including a body 1410 for positioning within an oral cavity of the user, the body 1410 defining at least one extra-oral opening 1431 for allowing airflow between lips of the user in fluid communication with at least one intra-oral opening (not shown) provided in the oral cavity to direct airflow into and/or out of a posterior region of the oral cavity. It will therefore be appreciated that this is broadly similar to the arrangement described above and further details will not therefore be described.

In this example, the intra-oral opening 1431 includes two individual intra-oral openings 1431.1, in fluid communication with intra-oral openings, via respective channels, whilst the intra-oral opening 1431 may be open to allow natural breathing directly through the opening.

In this example, the intra-oral opening 1431, which is formed from a tubular body that projects between the user's lips, also acts as a connector, allowing a connector system 1480 to be connected to the oral appliance for receiving air and/or oxygen from a positive airway pressure (PAP) device. By connecting a PAP device such as a continuous positive airway pressure (CPAP) machine to the oral appliance through the connector system, pressure and/or flow can be delivered directly to the oropharynx thereby bypassing obstructions from the nose, soft palate and tongue which can lead to snoring and apnoea events. The system is therefore able to operate at a much lower pressure than a traditional CPAP/mask combination which needs to supply sufficient pressure to open the obstructed airway of the user.

In this manner, the PAP device can be used as a source of pressure and/or flow to supplement natural breathing. For this reason, lower airflow is required from the PAP device compared to a traditional CPAP/mask combination that must provide substantially the entire air flow for the user. Whilst a standard CPAP machine may deliver around 70 litres/minute for breathing at rest, it is expected that flow rates of between 4 to 12 litres/minute would be suitable for a PAP device used in conjunction with the above-described oral appliance. Accordingly, it is to be appreciated that in the above described system, the pressure and flow requirements of a PAP device will be much lower than is currently the case for traditional CPAP/mask systems.

This in turn enables smaller PAP devices to be used, with smaller and less energy intensive pumps that will also reduce noise leading to increased patient comfort and compliance. The PAP devices may also be battery operated making them portable and more convenient for use.

The system may therefore be used for the treatment of sleep apnoea in all severities of patients who require to be treated with positive airway pressure and/or to receive supplemental air and/or oxygen.

The lower pressures and air flow required also overcomes issues with leakage experienced with a mask and the system is essentially maskless which further leads to increased comfort (e.g. no claustrophobia) and compliance. Additionally, minimal leakage also means lower air pressure and airflow is required to achieve a desired airway pressure.

In other arrangements, a connector system 1490 may be connected to the at least one extra-oral opening for natural breathing. As will be described in more detail below, one or more sensors such as position, temperature, airflow or pressure sensors may be located within the connector system to monitor body or head position, air temperature as well as flow rate and pressure on inhalation and exhalation through the oral appliance. Such sensor data may be used in a feedback control system which in one example may selectively switch on and off a PAP device so that pressure and/or flow is only provided as needed.

In the current example, the oral appliance 1400 has a pair of spaced apart first extra-oral openings 1431.1 that protrude from the front of the appliance to facilitate connection with the connector system 1480. A second extra-oral opening 1431.2 can be provided at the front of the appliance for allowing natural breathing and/or exhalation.

Each first extra-oral opening 1431.1 directs air through a channel that defines a first airway to a first intra-oral opening. The second extra-oral opening 1431.2 directs air through a channel that defines a second airway to a second intra-oral opening. The first and second airways can be separated by a partition so that independent airways are provided for PAP assisted and natural breathing.

The connector system 1480 can include a mouth connector 1481 including a body 1481.1 for connection to the oral appliance. An inlet chamber 1482 having an inlet 1482.1 for receiving a flow F of air from a PAP device (not shown) is connected to the mouth connector 1481. The mouth connector 1481 includes a pair of outlets 1481.2 that connect to the pair of first extra-oral openings 1431.1. In this way, air from the PAP device can be directed into the oral appliance 1400 through the first extra-oral openings 1431.1. Air from the PAP is then directed along the first airway to the first intra-oral opening where it is directed into the posterior region of the oral cavity proximate the oropharynx.

The inlet chamber 1482 can include an inlet valve 1482.2 for controlling airflow into the connector 1481. Any suitable type of valve may be used including for example a butterfly valve.

The mouth connector 1481 is further connected to a nasal connecting portion 1484 via a restrictor/valve in nasal inlet chamber 1483. The nasal connecting portion 1484 has a pair of outlets 1484.2 through which air from the PAP device is able to be delivered into the nose of the user. Optionally, nasal pillows or the like may be inserted into the nasal outlets 1484.2 for insertion into the user's nose.

In the above described system, air flow and/or pressure is able to be delivered from a PAP device, through passageways in the connector system to the oral appliance inlets 1431.1 and/or the nasal airway, to supplement natural breathing through the appliance 1400 which minimises airflow required to be delivered to the user. Furthermore, as air from the PAP is able to be delivered directly to the oropharynx bypassing obstructions from the nose, soft palate and tongue, less pressure is required to maintain an airway compared to a tradition CPAP/mask combination. Furthermore, the partitioned airway in the appliance enables a separate airway to be provided for exhalation which reduces effort on exhalation and as such increases comfort. In this instance, the exhalation airway would typically be provided to a respective first opening, such as the opening 1431.2.

By providing appliance sensing devices in the passageways within the connector system and within the oral appliance, this allows the system to monitor delivery of PAP to the user, allowing the effectiveness of the PAP system to be monitored.

As an alternative, a connector system 1490 can be attached to the appliance 1400, as shown in FIG. 14D. In this example, the connector system 1490 includes an inlet in the form of a vent that allows natural breathing through the second extra-oral openings 1431.2. The connector system 1490 may include a valve/restrictor, for example to control exhalation and/or a heat and moisture exchanger for controlling the water and temperature content of the air being inhaled by exchanging heat and moisture with exhaled air. In other arrangements, one or more sensors such as airflow or pressure sensors may be located within the connector system to monitor flow rate and pressure on inhalation and exhalation through the oral appliance.

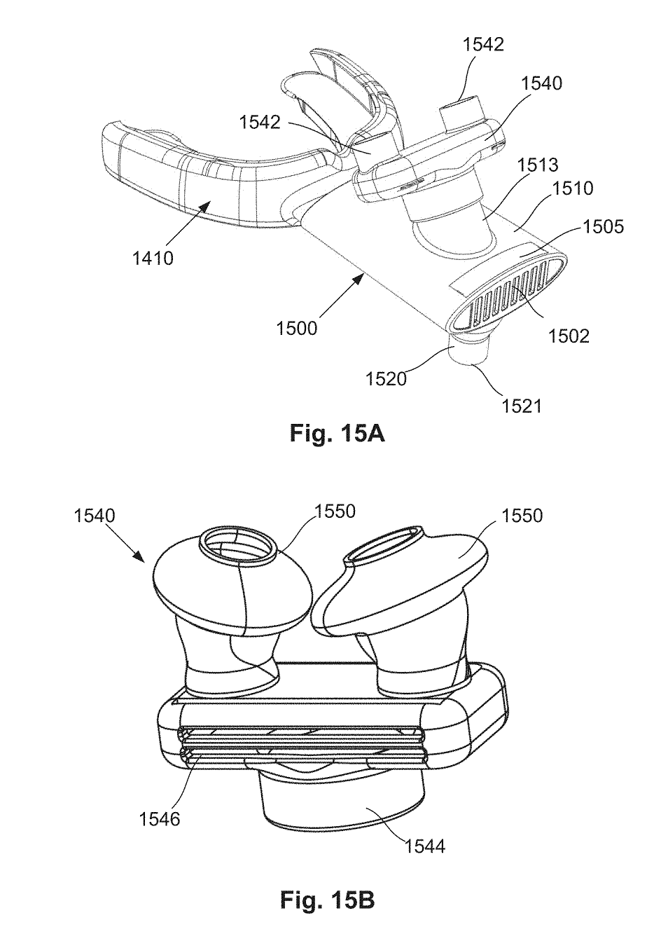

A further example of a connector system is shown in FIGS. 15A and 15B. In this example, the system includes an oral appliance substantially as described in co-pending Application No. PCT/AU2017/050271 having a body defining a single extra-oral opening in fluid communication with respective intra-oral openings via channels which define a dual airway for directing airflow to the posterior region of the oral cavity. The respective airways extend at least partially along the buccal cavity and at least partially between the teeth of the user in use.

The connector system 1500 includes a connector 1510 having a body of elliptic section with an opening 1512 that is complementary to the profile of the extra-oral opening of the appliance 1410 for connection thereto. Depending downwardly from the body 1510 is an inlet chamber 1520 having an inlet 1521 for allowing air from a PAP device into the inlet chamber 1520. The inlet chamber 1520 extends through the connector body 1510 and transitions into a nasal inlet chamber 1513 that projects away from the body 1510. In the example shown, the nasal inlet chamber 1513 is inclined relative to a direction of elongation of the connector body 1510. A nasal connecting portion 1540 is connected to the nasal inlet chamber 1513, the nasal connecting portion 1540 having a pair of outlets 1542 for directing air from the PAP device into the nasal cavity of the user.

At a distal end of the connector body 1510 is a breathing port or vent 1502 having an adjustable valve for allowing easy intake of air and controlled exhalation through the appliance. Additionally, a heat and moisture exchanger (HME) 1505 may also be provided for controlling the water and temperature content of the air being inhaled by exchanging heat and moisture with exhaled air. In one example, the heat and moisture exchanger may also act as the one-way valve. In such an arrangement, the heat and moisture exchanger may comprise a flap of material that is hingedly connected inside the connector body 1510 proximate the vent 1502. In use, as the patient breathes in, the flap pivots away from the vent 1502 to allow airflow into the appliance. When the user exhales, the flap pivots back towards the vent to substantially close the flow path thereby creating resistance upon exhalation. The level of resistance to exhalation may be controlled in any suitable manner including by providing one or more holes in the flap of HME material to provide a flow path for the expired air. It is to be understood that a one-way valve formed of HME material may also be implemented in any of the previously described examples.

Typically, the user is able to breathe naturally through the appliance with air flow travelling from vent 1502 (through valve and/or HME) through the connector body 1510 into the appliance then through extra-oral opening and along the airway to intra-oral openings where it is directed into a posterior region of the oral cavity. Meanwhile, nasal PAP can be delivered into the user's nose from the PAP device via the flow path formed by the tubing that extends through the connector body. In this regard, it is to be understood that in this example PAP is not delivered through the oral appliance and only to the nasal cavity of the user. The connector body 1510 is therefore used as a convenient means to secure the nasal PAP connector system to the oral appliance.

In FIG. 15B, there is shown an example of a nasal connecting portion 1540 having a pair of nasal pillows 1550 configured for insertion into the user's nostrils. The nasal pillows 1550 are typically made from a thermoplastic material that are custom heat moulded to suit a particular patient. After the nasal pillows 1550 are heat set and bent to shape they may then be cut to appropriate length. In this way, the nasal pillows 1550 are able to be customised to provide optimal comfort and cushioning when inserted into a user's nostrils. As an alternative to a thermoplastic material, the nasal pillows may be formed from any suitable flexible tubing that is able to be bent and shaped as needed. To assist the flexible tubing in maintaining shape, the wall structure of the tubing may include ductile metal strips or coil that is easily bent but provides additional stiffness to the tubing. The nasal pillows 1550 may be sleeved over the outlets 1542 shown in FIG. 7A. The nasal connecting portion 1540 further includes an adaptor portion 1544 for engagement with the nasal inlet chamber 1513. The nasal connector 1540 may also be adjustably positioned relative to the connector system 1500 to provide a customised fit and seal for the patient. One or more vents 1546 may also be provided in the body of the nasal connecting portion 1540.

Again, it will be appreciated that sensor arrangements similar to those described above can be incorporated into the connector system, allowing respiration and operation of the PAP device to be monitored.

In the above described example, the connector system 1500 is connected to the extra-oral opening of an oral appliance. However, this is not essential and an in alternative example, the connector system 1500 can be integrally formed with an oral appliance to effectively function as the extra-oral opening. In one example, the connector system 1500 can be in fluid communication with airways extending through the oral appliance, so that airflow is into a rear of the oral cavity. However, this is not essential and alternatively the connector system 1500 can simply pass between the lips of the user and open into a front of the user's oral cavity, allowing the user to perform the equivalent of open mouth and/or nasal breathing. In this instance, the oral appliance can effectively be a tray that cooperates with the user's teeth in order to hold the oral appliance in position in use and examples of this will be described in more detail below.

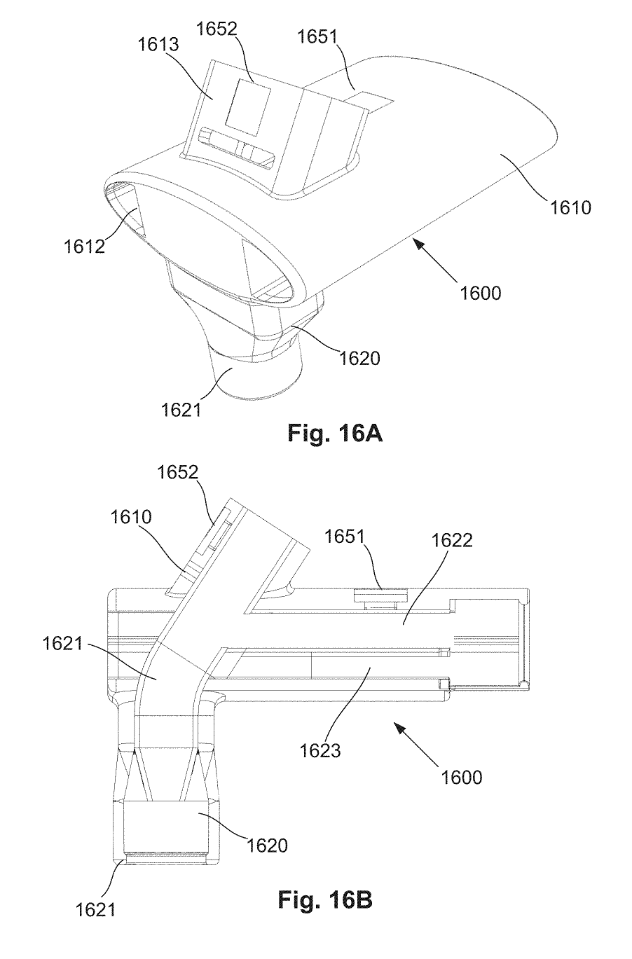

A further example of a connector system including monitoring devices is shown in FIGS. 16A and 16B. In this example, the connector system 1600 includes a connector 1610 having a body of elliptic section that is complementary to the profile of the extra-oral opening of the appliance for connection thereto, and with openings 1612 that communicate with first openings, such as the openings 1431.1, via a passageway 1623. Depending downwardly from the body 1610 is an inlet chamber 1620 having an inlet 1621 for allowing air from a PAP device into the inlet chamber 1620. The inlet chamber 1620 extends via a passageway 1621 through the connector body 1610 and transitions into a nasal inlet chamber 1613 that projects away from the body 1610. In the example shown, the nasal inlet chamber 1613 is inclined relative to a direction of elongation of the connector body 1610. A nasal connecting portion (not shown) can be connected to the nasal inlet chamber 1613 in a manner similar to that described above. The passageway 1621 is also connected to an oral passageway 1622 extending through the body 1610.

Typically, the user is able to breathe naturally through the appliance with air flow travelling from through the openings 1612 and passageway 1623 into the appliance then through extra-oral opening and along the airway to intra-oral openings where it is directed into a posterior region of the oral cavity. Meanwhile, nasal and oral PAP can be delivered into the user's nose and oral cavity from the PAP device via the flow path formed by the passageways 1621, 1622 that extend through the connector body. In this regard, it is to be understood that in this example PAP is delivered through both the oral appliance and the nasal cavity of the user.

In this example, two monitoring devices 1651 and 1652 are mounted in apertures extending through an upper surface of the body 1610 and a forward surface of the inlet 1621. Air pressure sensors are mounted so that these are in fluid communication with the nasal inlet chamber and oral passageway 1622, thereby allowing an oral and nasal PAP pressure to be measured using the process described above.

In this example, the connector system could be connected to an oral appliance including a body positioned in the mouth, and which includes a vent to allow natural breathing with the mouth open, to allow air to be delivered via intra-oral openings. Again, the connector system 1600 could alternatively be integrally formed with the oral appliance and examples of this will be described in more detail below.

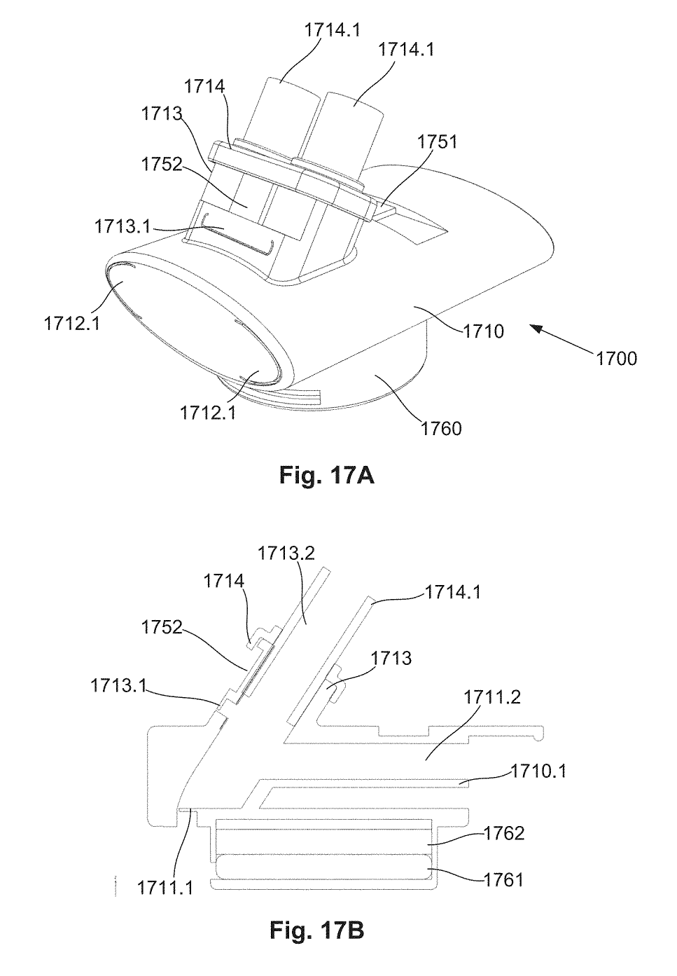

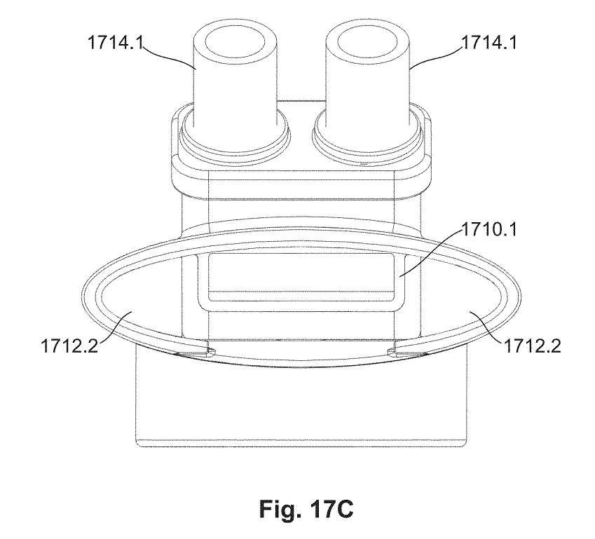

A further example connector system will now be described with reference to FIGS. 17A to 17C.

In this example, the connector system is used to connect monitoring electronics to the oral application, allowing respiratory characteristics to be measured for the user.

In this example, the connector system includes a connector body 1700 having an elliptic section that is complementary to the profile of the extra-oral opening of the appliance for connection thereto, and with first openings 1712.1 in a front of the body 1710 that communicate with respective internal passageways 1712.2 to provide a first oral flow path. The body 1710 includes an internal wall 1710.1 defining a second passageway 1711.2 in communication with a second opening 1711.1 on an underside of the body to define a second oral flow path.

The body 1710 includes a nasal support 1713, having a nasal connector 1714 slidably mounted thereto, allowing nasal prongs 1714.1, which in use are mounted to nasal pillows (not shown), to be positioned so that the nasal pillows engage the user's nasal airway. The nasal support 1713 includes a third opening 1713.1 mounted in a front side wall, which is in communication with a third passageway 1713.2 that defines a nasal flow path. In this example the second oral and nasal flow paths are in fluid communication, although this is not essential and these could alternatively be independent.

In this example, each of the openings includes a flow control valve in the form of a flap valve, that can be used to control inhalation and exhalation. In particular, providing the independent first and second oral flow paths, and through the use of appropriate valves, this allows for easy inhalation and more difficult exhalation, which ensures adequate flow of air into the user's oral and nasal airways, whilst maintain airway pressure during exhalation, which can in turn help prevent airway collapse.

In this example, the appliance monitoring device is formed from first and second air pressure sensors 1751, 1752 mounted in the body 1710 and the nasal support 1713, electronics 1762, including a processing device and data store, and a battery 1761, mounted in a housing 1760 on an underside of the connector body 1710. In this example, the housing 1760 includes a slit allowing the battery to be replaced as required.

In this example, each of the air pressure sensors 1751, 1752 are in fluid communication with the second oral passageway and the nasal passageway, via respective openings in the body 1710 and the nasal support 1713, allowing air pressures therein to be sensed. The sensors 1751, 1752 are electrically connected to the processing device, allowing signals from the sensors to be recorded and stored prior to transfer to a separate processing system, as previously described. Again, the connector system 1700 could alternatively be integrally formed with the oral appliance and examples of this will be described in more detail below.

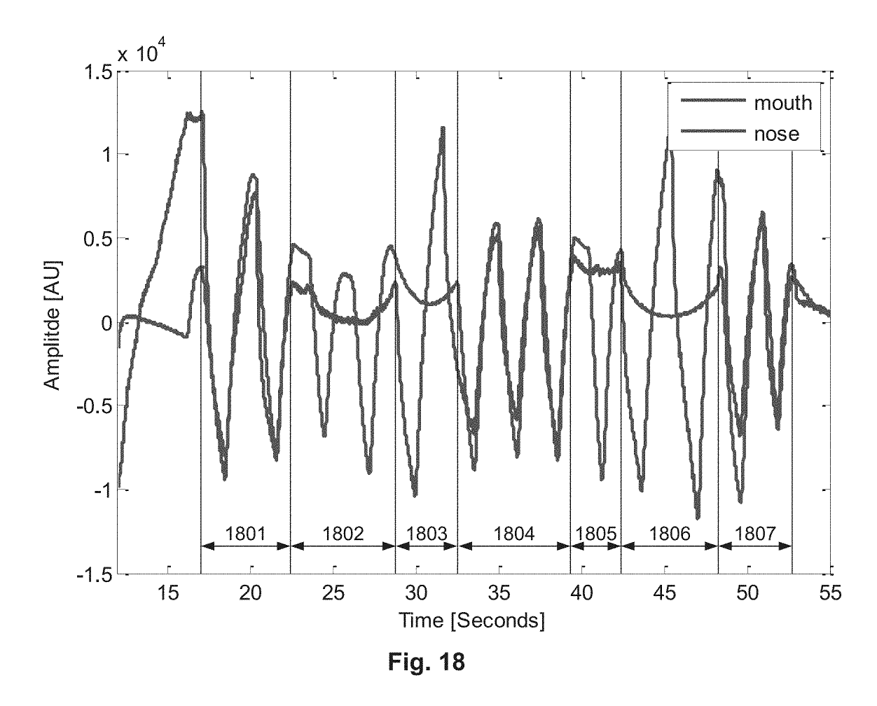

An example of the air pressure changes recorded during breathing, including the relative nasal and second oral passageway pressures, are shown in FIG. 18.

In this example, a number of different types of breathing are shown, including:
Balanced oral and nasal breathing 1801 and 1807
Nasal only breathing 1802 and 1805
Oral only breathing 1803 and 1806
Nasal preferential breathing 1804

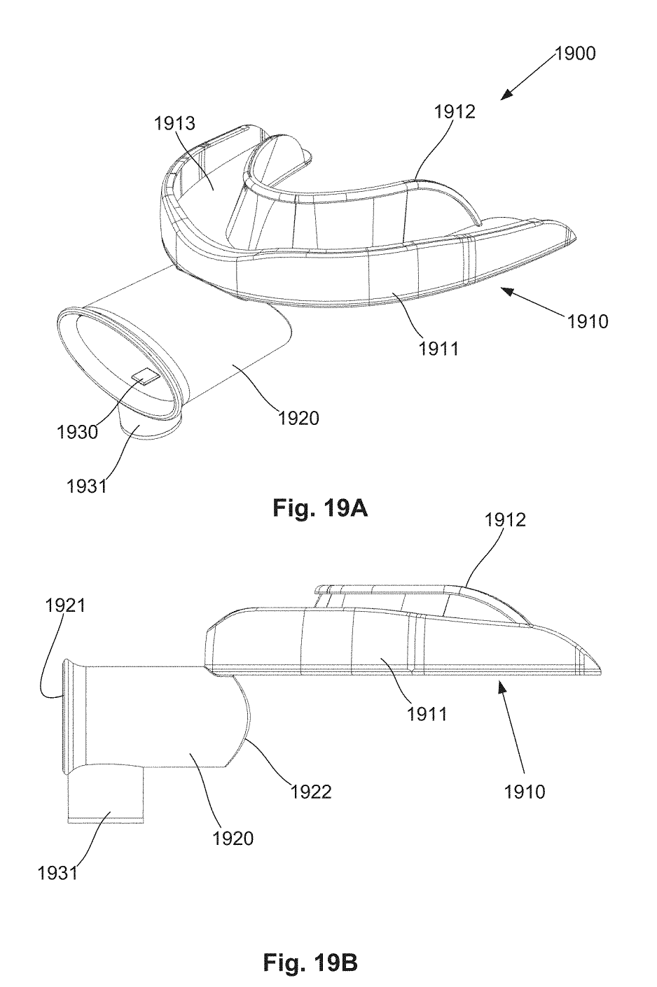

A further example of an oral appliance including an integral connector system will now be described with reference to FIGS. 19A and 19B.

In this example, the oral appliance 1900 includes a body 1910, which is in the form of a tray, or similar. The tray typically includes spaced apart arcuate front and rear side walls 1911, 1912 extending upwardly from a planar base 1913, to define a recess that can accommodate a user's teeth, or a bite member which in turn receives the user's teeth. In this current example, the body 1910 is adapted to be received by the user's maxillary teeth, but this is not essential and alternatively the tray could be upturned so as to receive the mandibular teeth.

The body 1910 is coupled to or integrally formed with a connector system defined by a generally elliptically cross sectioned hollow tube 1920, which in use extends from a front of the tray to thereby pass between lips of the user in use. The connector system 1920 defines an extra-oral opening 1921 and intra-oral opening 1922 connected by an internal oral passageway defined by the tube 1920, allowing air to flow through the device between the user's lips, to thereby enable inhalation and exhalation through the oral passageway.

In this example, the connector system includes an appliance monitoring device having an air pressure sensor 1930 mounted in an internal side wall of the tube 1920, allowing changes in air pressure therein to be detected. Additional components, including a battery and electronics can be contained in a housing extending downwardly from an outer surface of the tube 1920. In this instance, the electronics are mounted on a surface opposite pressure sensor 1930, so the pressure sensor can be directly connected. However, it will be appreciated that this is not essential, and alternatively the air pressure sensor 1930 and electronics can be provided at different locations on the tube 1920, and electrically connected as required.

In the current example, the tube has a substantially constant diameter along the length of the tube, with a slight flaring at the opening 1921. However, this is not essential, and alternatively inside of the tube 1920 can be shaped so as to maximise airflow over the sensor. This can be achieved through any suitable arrangement, such as through suitable flaring of the tube, positioning of the sensor adjacent a flow restriction, such as a narrowing of the tube or baffle, or the like.

Accordingly, it will be appreciated that the above described system provides a simple oral appliance that can be used in monitoring breathing of a user, for example, allowing oral inhalation and exhalation to be measured.

In one example, as no breathing additional assistance, such as mandibular advancement or an additional airway to the rear of the user's oral cavity, is provided, this can be useful in performing a base-line sleep test.

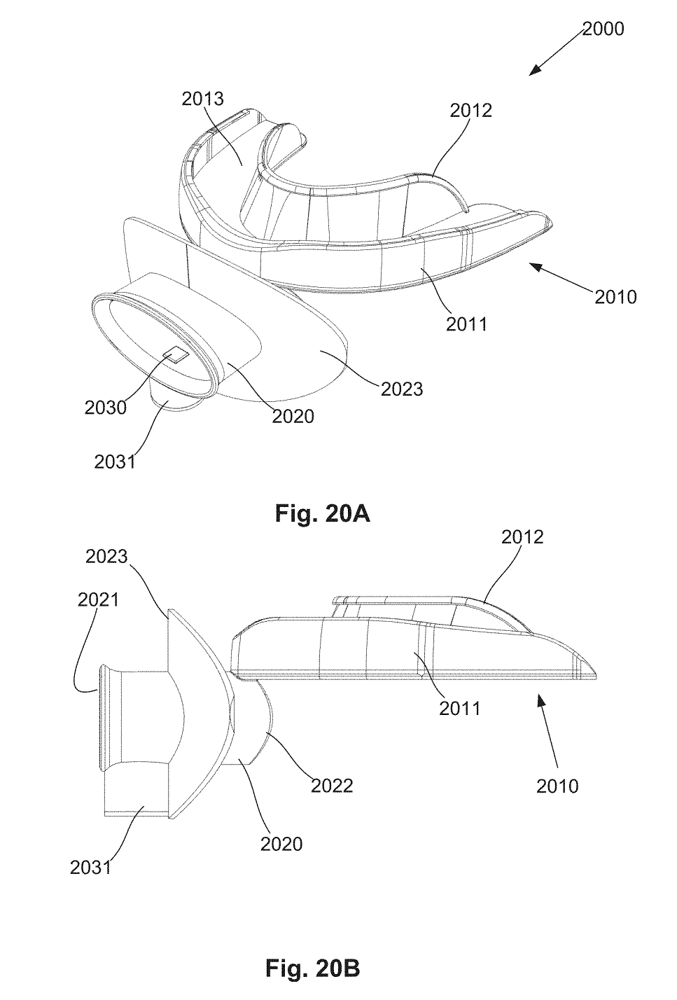

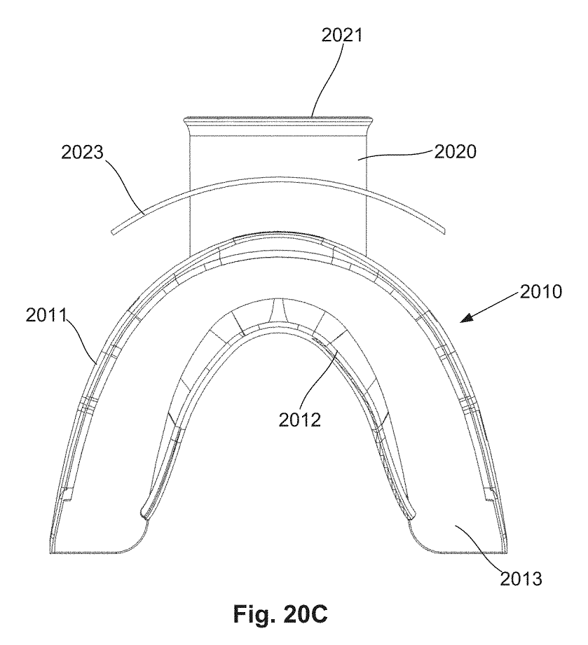

A further example of an oral appliance including an integral connector system will now be described with reference to FIGS. 20A to 20C. This example is substantially similar to that described above with respect to FIGS. 19A and 19B, and similar reference numerals increased by 100 are used to denote similar features, which will not therefore be described in further detail.

In this example, the connecting system includes additional wings 2023, extending outwardly from the tube 2020. The wings are arcuately shaped and designed to rest against an outer surface of the user's lips to cover the mouth and ensure that most of the air being breathed in orally goes past the sensor. The wings can be integrally formed with the connector system 2020, or could be a separate, flexible cover adjustably mounted to the tube 2020, or even separately mounted and held in place by straps extending around the user's head.

In the above example, the connector system tube 1920 can be fixed to the body 1910, or could be adjustable mounted thereto, allowing this to be moved longitudinally relative to the body, so as to adjust the positioning of the wings. Furthermore, whilst wings are shown extending upwardly and downwardly from the tube 202, this is not essential, and alternatively the wings could extend from the top or bottom only.

In any event, it will again be appreciated that the arrangement of FIGS. 20A to 20C can be used to assist in monitoring breathing of the user, and can be used in a variety of circumstances, such as performing a base line sleep test, or the like.

Accordingly, it will be appreciated that the above described arrangements provide mechanisms for monitoring oral appliance compliance, and for monitoring breathing as well as operation of assist devices. This is in contrast to current methodologies that are typically based upon the combined inertial position of the sensor and stabilization of the device's temperature to body temperature when worn for a period of time. The disclosed invention instead uses combined information from device specific and physiological parameters to detect the time epoch for the monitoring.

In one example, the sensing arrangement can measure device specific parameters including absolute pressure (force over area of the sensor) spike generated by the insertion of the oral appliance under monitoring and a content or touch sensor that is activated when any part of the body is in contact with the sensor. The system can further measure physiological parameters, including pressures and temperatures. In this regard, once the device is worn, absolute pressure will fluctuate with the respiration around the value measured at the “spike” and superior to the atmospheric pressure due to the weight of the anatomical part in contact with the device, whilst the temperature will remain quasi-steady (may fluctuate with respiration) around the body temperature.

In one example, the contact sensor or “touch toggle” is configured to wake the device and put it in the pre-monitoring state. In pre-monitoring stage the device await a change in environmental/physiological quantities to confirm the start of the monitoring. The use of combined physiological and device specific parameters minimizes errors and give precise time epochs.

Accordingly, the above described arrangement can operate to measure compliance. In one example, this uses a sensor that measures temperature and pressure. The sensor(s) may be located in a region of the appliance to measure downward, forward or backward pressure from the teeth or lips, for example in the bite member or on or in the extra-oral opening to measure lip pressure and lip temperature. This latter arrangement can also be used in order to measure airflow and/or snoring. Sensors are associated with a CPU and battery to allow recording of the data for each defined interval.

The monitoring device can then be periodically connected to a computer or smart phone app for downloading data which can then be graphed and analysed. This can be performed locally and/or in a HIPAA (Health Insurance Portability and Accountability Act of 1996) compliant cloud based system.

In one example, the monitoring device could be integrally formed as part of the appliance. Alternatively, the monitoring device can be provided in a moulded component that can be removed for replacement or cleaning. In one example, this is achieved using a slot in the body of the intra-oral opening, within the monitoring device slides in and clipping into place. A blank moulded component can be used instead if the compliance assembly is an optional extra to the appliance. It could also be removed for download of data and/or cleaning the appliance.

The system can also be used to measure airflow within an airway. This could include an airway of the appliance and/or the airway of a connector system coupled to the appliance. In this regard, the appliance can be used with a connector system that provides one or more airways for connection to a PAP. In this case, the monitoring device could include one or more sensors that measure airflow mounted in set locations in the connector system. This may be in the extra-oral opening oral flow or in the nasal region of the connector for nasal flow.

The system can also be used with oral appliance that does not otherwise alter airflow, such as by offering mandibular advancement or airways to the back of the mouth, allowing this to be used as part of a baseline sleep test/monitoring. In this case, the extra-oral opening at the front of the appliance would open directly into the front of the mouth, and would include a connector with a sensor which monitors oral breathing. An additional sensor could be mounted in a port that goes from the extra-oral opening to the front of the mouth to detect open mouth breathing. The air would then flow to the extra-oral opening. A “snorkel” like attachment that the user bites on for mouth breathing could also be employed, with this being used to allow air to pass to the extra-oral opening to detect inhalation as well as exhalation. These designs will allow the extra-oral opening to stay in the same relative position to the nose for the control device.

It will be appreciated that the arrangements used for compliance and airway monitoring are largely the same, although the sensor for airway monitoring may not be embedded or otherwise sealed in a polymer as this step may restrict airflow being detected. For airway sensing additional processing and data recording may be required, in which case faster, more regular detection may be performed, with a larger power supply being used as required. In this case, the monitoring device including the CPU, battery and antennae can be housed in a plastic case underneath the connector which is integrally moulded with the connector itself.

In addition to pressure and temperature sensing, additional sensors could also be employed depending on the preferred implementation. For example, this could include the use of an accelerometer that can monitor head position.

To complete a level III sleep test there would need to be oximetry and heart rate detection which can be provided in a separate stand-alone user monitoring device, with a respective processor and transmitter. Data could then be downloaded to the same processing system, such as a smart phone app and/or a computer, and analysed in conjunction with sensor data from the appliance monitoring device. The user monitoring device could be wrist or ear mounted depending on the preferred implementation. If the wrist position of the arm cannot be determined however it would have closer proximity to the head for EEG detection for a level II sleep test. If a level II system is required, then ECG and EEG would need to be added to the system. There is also a possibility to measure EEG from the inner hear using an aural insert.

Accordingly, it will be appreciated that the monitoring device can be used in conjunction with other monitoring devices in order to provide for clinical evaluation during sleep studies. In this example, an app completes the sleep diagnosis and sleep monitoring system.

In general, results of compliance and/or airway monitoring can be displayed via an app on a user's smartphone or other similar device. The app can be configured to show a wide range of information, including but not limited to:
Compliance through the night by graphing pressure/temperature vs time
Apnoea and hypopnea events from airflow
Blood oximetry and heart rate vs time
Head position vs time with
Body Position from wrist accelerometer vs time
Correlation of these to compliance eg with red lines on graphs in regions where the appliance is not worn
AHI and O₂ saturation correlation with both compliance and position

It will be appreciated that this can be used to provide a sleep monitoring system that does not have the capital equipment requirements or cost of the normal overnight sleep test equipment. The system can be used multiple nights for more accurate and more regular monitoring, and is capable of measuring both nasal and oral airflow for more accurate estimation of AHI.

In one example, the monitoring system can be included in the cost of the appliance, with costs being recovered as part of the process of having sleep clinicians paid to report on the data following a referral by the appropriate clinician. The compliance sensor allows the recording and monitoring of when the appliance is being used by the patient. This is important for the sleep clinician/dentist to optimise therapy and also for payors to justify payment.

The system can also be connected to a CPAP to provide biofeedback for more controlled delivery of the positive (or negative) airway pressure in an on or off mode. When a PAP is being used the wires for the airflow and/or pressure sensors can be embedded in or coiled around the CPAP hose to the CPU/power supply located in or on the CPAP itself. This means the CPU, battery and Bluetooth antennae are not needed in the connector itself.

Primarily the sleep system allows the monitoring of patients while asleep. The sleep test electronics as well as the sensors are in a connector system that fits on the front of an oral appliance and also has airways to the patient's nose to monitor nasal breathing.

The system can be used in a variety of configurations, including but not limited to:
In an appliance with no breathing assistance, including no mandibular advancement or internal airway
A single sensor for mouth breathing through the appliance
A dual sensor for mouth breathing and nasal breathing
Mouth breathing plus nasal PAP
Mouth PAP plus nasal PAP

The versions connected to PAP can have the sensor mounted in the PAP device itself or in the connector system used to connect the PAP device to the appliance. Outputs from the sensors can also be used to control the PAP based on biofeedback.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means±20%.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1) A system for monitoring use of an oral appliance, the oral appliance including a body that is positioned within an oral cavity of the user in use, the system including:
a) an appliance monitoring device which in use is attached to or embedded within the oral appliance, the appliance monitoring device including:
i) at least one sensor, the at least one sensor including a pressure sensor that generates a signal indicative of a pressure;
ii) a data store; and,
iii) a processing device that:
(1) analyses signals from the at least one sensor to determine a usage state;
(2) in response to a determination that the appliance is in use, generates usage data at least partially indicative of the use; and,
(3) stores the usage data in the data store;
b) one or more processing systems that:
i) acquire the usage data from the monitoring device; and,
ii) at least one of:
(1) store an indication of the usage data; and,
(2) cause a representation to be displayed at least partially in accordance with the usage data.

2) A system according to claim 1, wherein the appliance monitoring device includes at least one of:
a) a clock, the clock generating an indication of at least one of a time and a date;
b) a physical connection that connects to the one or more processing systems, the processing device operating to transfer the usage data to the one or more processing systems via the connection;
c) a transmitter, the processing device operating to transmit the usage data to the one or more processing systems using the transmitter; and,
d) a power supply that powers the at least one sensor and the processing device.

3) A system according to claim 1 or claim 2, wherein the appliance monitoring device includes a housing containing the at least one sensor and the processing device, the housing being removably mounted to the body.

4) A system according to claim 1 or claim 2, wherein the appliance monitoring device includes:
a) a sensor housing containing the at least one sensor;
b) a processing device housing containing the processing device; and,
c) at least one electrical connection extending between the sensor and processing device housings.

5) A system according to claim 3 or claim 4, wherein the appliance monitoring device includes a housing mounted to the body such that the housing is outside of the oral cavity in use.

6) A system according to any one of the claims 1 to 5, wherein the pressure sensor measures at least one of:
a) an air pressure in an airway of the oral appliance;
b) an air pressure in an airway of the user;
c) a contact between the user and the appliance; and,
d) a contact pressure of contact between the user and the appliance.

7) A system according to any one of the claims 1 to 6, wherein the at least one sensor includes:
a) a first pressure sensor that measures at least one of a contact or contact pressure;
b) a second pressure sensor that measures an air pressure in an airway;
c) an oxygen sensor that senses oxygen levels in exhaled air;
d) a carbon dioxide sensor that senses carbon dioxide levels in exhaled air;
e) a temperature sensor that measures a temperature;
f) a moisture sensor that measures a moisture;
g) a humidity sensor that measures a humidity; and,
h) a movement sensor that measures at least one of:
i) a position of the oral appliance;
ii) an orientation of the oral appliance; and,
iii) a movement of the oral appliance.

8) A system according to any one of the claims 1 to 7, wherein the usage data includes at least one of:
a) an identifier indicative of at least one of an identity and type of the oral appliance;
b) a time the signals were measured;
c) a date on which the signals were measured;
d) an indication of a usage state;
e) sensor data indicative of signals from the sensors;
f) at least one parameter at least partially derived using signals from the sensors;
g) compliance data indicative of a compliance period associated with a period of use of the oral appliance by a user;
h) respiratory device data indicative of at least one operational characteristic of a respiratory device;
i) respiratory data indicative of at least one respiratory characteristic of the user of the oral appliance; and,
j) sleep data indicative of at least one sleep characteristic of the user of the oral appliance.

9) A system according to any one of the claims 1 to 8, wherein the representation is indicative of at least one of:
a) signals from the at least one sensor;
b) changes in at least one parameter over time;
c) comparison of the signals to one or more thresholds; and,
d) comparison of the at least one parameter to one or more thresholds.

10) A monitoring system according to any one of the claims 1 to 9, wherein the monitoring device at least partially processes the sensor signals by at least one:
a) filtering the signals;
b) amplifying the signals;
c) digitizing the signals; and,
d) parameterizing the signals.

11) A system according to any one of the claims 1 to 10, wherein the processing device determines if the appliance is in use based on a signal from a pressure sensor indicative of a contact between the user and the appliance.

12) A system according to claim 10, wherein:
a) if an appliance is in use, the processing device at least one of:
i) selectively updates compliance data indicative of a compliance period; and,
ii) records sensor data indicative of signals from the sensors.
b) if an appliance is not in use, the processing device:
i) uses signals from the at least one sensor to generate a reference; and,
ii) stores an indication of the reference in the data store.

13) A system according to claim 11 or claim 12, wherein if the appliance is in use, the processing device:
a) uses usage data to determine if a compliance period is underway; and,
b) if a compliance period is underway:
i) determines if at least one of a pressure determined by a pressure sensor and a temperature determined by a temperature sensor exceed a respective reference; and,
ii) in response to a successful determination, updates compliance data to extend the compliance period;
c) if a compliance period is not underway:
i) monitors for a change in at least one of a pressure determined by a pressure sensor and a temperature determined a temperature sensor; and,
ii) in response to a change, updates compliance data to commence a compliance period.

14) A system according to claim 13, wherein the change corresponds to at least one of:
a) spikes;
b) changes having a magnitude greater than a threshold; and,
c) changes having a rate of change greater than a threshold.

15) A system according to any one of the claims 11 to 14, wherein the processing device uses a usage state to at least partially control operation of the system.

16) A system according to claim 15, wherein the processing device uses the usage state to control a signal sampling rate.

17) A system according to claim 15 or claim 16, wherein the processing device:
a) exits a low power mode;
b) determines the usage state;
c) optionally generates usage data; and,
d) returns to the low power mode for a defined time interval.

18) A system according to claim 17, wherein the defined time limit is set in accordance with the usage state.

19) A system according to any one of the claims 1 to 18, wherein the system:
a) analyses sensor data from the at least one sensor to determine at least one of:
i) a temperature in an airway; and,
ii) an air pressure in an airway;
b) uses at least one of the temperature and air pressure to monitor at least one of:
i) respiratory device data indicative of at least one operational characteristic of a respiratory device;
ii) respiratory data indicative of at least one respiratory characteristic of the user of the oral appliance; and,
iii) sleep data indicative of at least one sleep characteristic of the user of the oral appliance.

20) A system according to any one of the claims 1 to 19, wherein the system analyses sensor data from the at least one sensor to determine at least one of:
a) a respiration rate;
b) a respiration magnitude; and,
c) a degree of snoring.

21) A system according to any one of the claims 1 to 20, wherein the system is used to perform a sleep test and wherein the pressure sensor includes an air pressure sensor that measures an air pressure in an airway of the oral appliance or a connector system, and wherein the sensor data is used to generate at least one of:
a) respiratory data indicative of at least one respiratory characteristic of the user of the oral appliance; and,
b) sleep data indicative of at least one sleep characteristic of the user of the oral appliance.

22) A system according to any one of the claims 1 to 21, wherein the one or more processing systems:
a) acquire user sensor data indicative of signals from at least one user sensor; and,
b) generate sleep data at least partially indicative of a sleep characteristic of a user of the oral appliance at least in part using the usage data and the user sensor data.

23) A system according to claim 22, wherein the at least one user sensor includes at least one of:
a) an oxygen sensor that senses oxygen levels in exhaled air;
b) a carbon dioxide sensor that senses carbon dioxide levels in exhaled air;
c) respiratory sensors that sense a respiratory effort or rate;
d) pulse oximetry sensor that measures a blood oxygen level;
e) an ECG sensor;
f) an EEG sensor; and,
g) a heart rate sensor that measures a heart rate.

24) A system according to any one of the claims 1 to 23, wherein the oral appliance includes at least one bite member coupled to the body, the bite member being positioned at least partially between the user's teeth and the body in use, and wherein the at least one sensor includes a pressure sensor that senses contact of the user's teeth with the bite member based on a pressure between the body and bite member.

25) A system according to any one of the claims 1 to 24, wherein oral appliance includes first and second bodies, the first body includes an adjustable mounting configured to interconnect the first and second bodies to thereby allow a relative position of the first and second bodies to be adjusted, and wherein the at least one sensor includes a pressure sensor configured to determine a relative pressure between the first and second bodies.

26) A system according to any one of the claims 1 to 25, wherein the oral appliance includes an extra-oral opening for allowing airflow between lips of the user and wherein the at least one sensor monitors at least one of an air pressure and a temperature in the extra-oral opening.

27) A system according to any one of the claims 1 to 26, wherein the oral appliance includes at least one extra-oral opening defined by a tubular body protruding from the appliance and wherein the at least one sensor includes a pressure sensor that senses a contact between a user's lips and an external surface of the tubular body.

28) A system according to any one of the claims 1 to 27, wherein the oral appliance includes at least one extra-oral opening in fluid communication with at least one intra-oral opening via a channel, the intra-oral opening being provided in the oral cavity to direct airflow into and/or out of a posterior region of the oral cavity, and wherein the at least one sensor monitors at least one of an air pressure and a temperature in the channel.

29) A system according to any one of the claims 1 to 28, wherein the body defines at least two channels, each channel connecting an intra-oral opening to at least one extra-oral opening, each channel passing at least one of at least partially along the buccal cavity and at least partially between the teeth to thereby provide an airway for the user, the airway at least partially bypassing the nasal passage and acting to replicate a healthy nasal passage and pharyngeal space.

30) A system according to any one of the claims 1 to 29, wherein the oral appliance includes at least one extra-oral connector and a connector system connected to the at least one extra-oral connector, the connector system including at least one passageway for allowing airflow through at least one of the oral appliance and nose of the user, and wherein the at least one sensor monitors at least one of an air pressure and a temperature in the at least one passageway.

31) A system according to claim 30, wherein the passageway is connected to a positive airway pressure (PAP) device, and wherein the at least one sensor monitors operation of the PAP device.

32) A system according to claim 30 or claim 31, wherein air from the PAP device is delivered to the user through at least one of:
a) the nose of the user; and,
b) the oral appliance.

33) A system according to any one of the claims 1 to 32, wherein the oral appliance includes at least one of:
a) a valve;
b) a restrictor; and,
c) a heat and moisture exchanger.

34) A system according to any one of the claims 1 to 33, wherein the oral appliance includes a number of ports to allow for inhalation or exhalation, wherein at least one port includes a valve to control a flow restriction.

35) A system according to any one of the claims 1 to 34, wherein the system includes at least two air pressure sensors for measuring nasal and oral breathing respectively.

36) A system breathing assistance system including:
a) an oral appliance, the oral appliance including a body that is positioned within an oral cavity of the user in use;
b) an appliance monitoring device which in use is attached to or embedded within the oral appliance, the appliance monitoring device including:
i) at least one sensor, the at least one sensor including a pressure sensor that generates a signal indicative of a pressure; and,
ii) a data store; and,
iii) a processing device that:
(1) analyses signals from the at least one sensor to determine a usage state;
(2) in response to a determination that the appliance is in use, generates usage data at least partially indicative of the use; and,
(3) stores the usage data in the data store;
c) one or more processing systems that:
i) acquire the usage data from the monitoring device; and,
ii) at least one of:
(1) store an indication of the usage data; and,
(2) cause a representation to be displayed at least partially in accordance with the usage data.

37) An appliance monitoring device which in use is attached to or embedded within an oral appliance, the appliance monitoring device including:
a) at least one sensor, the at least one sensor including a pressure sensor that generates a signal indicative of a pressure; and,
b) a data store; and,
c) a processing device that:
i) analyses signals from the at least one sensor to determine a usage state;
ii) in response to a determination that the appliance is in use, generates usage data at least partially indicative of the use; and,
iii) stores the usage data in the data store.

38) A method for monitoring use of an oral appliance, the oral appliance including a body that is positioned within an oral cavity of the user in use, the method including:
a) providing an appliance monitoring device which is attached to or embedded within the oral appliance, the appliance monitoring device including:
i) at least one sensor, the at least one sensor including a pressure sensor that generates a signal indicative of a pressure; and,
ii) a data store; and,
iii) a processing device;
b) using the processing device to:
i) analyse signals from the at least one sensor to determine a usage state;
ii) in response to a determination that the appliance is in use, generate usage data at least partially indicative of the use; and,
iii) store the usage data in the data store;
c) using one or more processing systems to:
i) acquire the usage data from the monitoring device; and,
ii) at least one of:
(1) store an indication of the usage data; and,
(2) cause a representation to be displayed at least partially in accordance with the usage data.

39) A system for monitoring a user of an oral appliance, the oral appliance including a body that is positioned within an oral cavity of the user in use, the system including:
a) an appliance monitoring device which in use is attached to or embedded within the oral appliance, the appliance monitoring device including:
i) at least one sensor, the at least one sensor including an air pressure sensor that generates a signal indicative of an air pressure;
ii) a data store; and,
iii) a processing device that:
(1) receives signals from the at least one sensor; and,
(2) stores sensor data indicative of signals from the sensors in the data store;
b) one or more processing systems that:
i) acquire the sensor data from the appliance monitoring device; and,
ii) use the sensor data to generate at least one of:
(1) respiratory data indicative of at least one respiratory characteristic of the user of the oral appliance; and,
(2) sleep data indicative of at least one sleep characteristic of the user of the oral appliance.

40) A system according to claim 39, wherein the system includes a user monitoring device including at least user one sensor for monitoring an attribute of a user, and wherein the one or more processing systems:
a) acquire user sensor data indicative of signals from at least one user sensor; and,
b) generate sleep data at least partially indicative of a sleep characteristic of a user of the oral appliance at least in part using the sensor data and the user sensor data.

41) A system according to claim 40, wherein the at least one user sensor includes at least one of:
a) an oxygen sensor that senses oxygen levels in exhaled air;
b) a carbon dioxide sensor that senses carbon dioxide levels in exhaled air;
c) respiratory sensors that sense a respiratory effort or rate;
d) pulse oximetry sensor that measures a blood oxygen level;
e) an ECG sensor;
f) an EEG sensor; and,
g) a heart rate sensor that measures a heart rate.

42) A system according to any one of the claims 39 to 41, wherein the system is used to perform a sleep test.

43) A method for monitoring a user of an oral appliance, the oral appliance including a body that is positioned within an oral cavity of the user in use, the method including:
a) providing an appliance monitoring device which in use is attached to or embedded within the oral appliance, the appliance monitoring device including:
i) at least one sensor, the at least one sensor including an air pressure sensor that generates a signal indicative of an air pressure;
ii) a data store; and,
iii) a processing device;
b) using the processing device to:
i) receive signals from the at least one sensor; and,
ii) store sensor data indicative of signals from the sensors in the data store;
c) using one or more processing systems to:
i) acquire the sensor data from the appliance monitoring device; and,
ii) use the sensor data to generate at least one of:
(1) respiratory data indicative of at least one respiratory characteristic of the user of the oral appliance; and,
(2) sleep data indicative of at least one sleep characteristic of the user of the oral appliance.
